SEQUENCE LISTING (34 sequences)

SEQ ID NO: 1 - protein, 346 aa, synthetic construct
  REGION 1..346: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSTNIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATGACNAGGFSWRRYRSAESVDKRATMTDAELEKKLNSYVQMDKEYVKNNQARS

SEQ ID NO: 2 - DNA, 1057 bp, synthetic construct
  misc_feature 1..1057: note = Polynucleotide encoding synthetic polypeptide
  source 1..1057: mol_type = other DNA
ggtctcgaatgcttctcctctccctcctctccattgtcaccctcgctgttgcttctcctctgtccgttgaggagtacgccaaggccctcgaggagcgtgccgtcaccgtctcctcctccgagctcaacaacttcaagttctacatccagcacggtgctgctgcctactgcaactccgagactgctgctggtgccaacgtcacctgcactggcaacgcctgccccgagattgaggccaacggtgtcaccgttgttgcctccttcactggtaccaagactggtatcggtggctacgtctccaccgacaacaccaacaaggagatcgtcctttctttccgtggcagcaccaacatccgcaactggctgaccaacctggacttcggccaggatgactgctctctgacctccggctgcggtgtccactccggtttccagcgtgcctgggaggagattgccgacaacctgaccgctgctgttgccaaggccaagactgccaaccccgactacaaggttgttgccactggccactccctgggtggtgctgttgccaccctggctggtgccaacctccgtgctgctggtacccccctcgacatctacacctacggctctccccgtgtcggcaacgccgagcttgctgagttcatctccaaccagactggtggtgagttccgtgtcacccacggtgatgaccccgtcccccgtcttcctcctctgatcttcggctaccgccacacctcccccgagtactggctcgatggcagcggtggtgacaagatcaactacaccatcaacgacatcaaggtctgcgagggtgctgccaacctgcagtgcaacggtggtaccctgggcctcgacattgctgctcacctgcactacttccaggccactggtgcctgcaacgccggtggtttcagctggcgccgctaccgctctgctgagagcgttgacaagcgtgccaccatgactgatgctgagctcgagaagaagctcaacagctacgtgcagatggacaaggagtacgtcaagaacaaccaggctcgctcctaaacgagacc

SEQ ID NO: 3 - protein, 346 aa, synthetic construct
  REGION 1..346: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSTNIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATGACNAGGFSWQRYRSAESVDKRATMTDAELEKKLNSYVQMDKEYVKNNQARS

SEQ ID NO: 4 - protein, 346 aa, synthetic construct
  REGION 1..346: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSTNIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATGACNAGGFSWARYRSAESVDKRATMTDAELEKKLNSYVQMDKEYVKNNQARS

SEQ ID NO: 5 - protein, 346 aa, synthetic construct
  REGION 1..346: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSTNIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATGACNAGGFSWRQYRSAESVDKRATMTDAELEKKLNSYVQMDKEYVKNNQARS

SEQ ID NO: 6 - protein, 346 aa, synthetic construct
  REGION 1..346: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSTNIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATGACNAGGFSWRAYRSAESVDKRATMTDAELEKKLNSYVQMDKEYVKNNQARS

SEQ ID NO: 7 - protein, 338 aa, synthetic construct
  REGION 1..338: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSTNIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATGACNAGGFSWKEREEDISDAELEKKLNSYVQMDKEYVKNNQARS

SEQ ID NO: 8 - protein, 338 aa, synthetic construct
  REGION 1..338: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSTNIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATGACNAGGFSWRQREEEISDAELEKKLNSYVQMDKEYVKNNQARS

SEQ ID NO: 9 - protein, 335 aa, synthetic construct
  REGION 1..335: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSTNIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATGACNAGGFSWKPHMSDAELEKKLNSYVQMDKEYVKNNQARS

SEQ ID NO: 10 - protein, 338 aa, synthetic construct
  REGION 1..338: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSTNIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATGACNAGGFSWRSRQEDISDAELEKKLNSYVQMDKEYVKNNQARS

SEQ ID NO: 11 - DNA, 1057 bp, synthetic construct
  misc_feature 1..1057: note = Polynucleotide encoding synthetic polypeptide
  source 1..1057: mol_type = other DNA
ggtctcgaatgcttctcctctccctcctctccattgtcaccctcgctgttgcttctcctctgtccgttgaggagtacgccaaggccctcgaggagcgtgccgtcaccgtctcctcctccgagctcaacaacttcaagttctacatccagcacggtgctgctgcctactgcaactccgagactgctgctggtgccaacgtcacctgcactggcaacgcctgccccgagattgaggccaacggtgtcaccgttgttgcctccttcactggtaccaagactggtatcggtggctacgtctccaccgacaacaccaacaaggagatcgtcctttctttccgtggcagcaccaacatccgcaactggctgaccaacctggacttcggccaggatgactgctctctgacctccggctgcggtgtccactccggtttccagcgtgcctgggaggagattgccgacaacctgaccgctgctgttgccaaggccaagactgccaaccccgactacaaggttgttgccactggccactccctgggtggtgctgttgccaccctggctggtgccaacctccgtgctgctggtacccccctcgacatctacacctacggctctccccgtgtcggcaacgccgagcttgctgagttcatctccaaccagactggtggtgagttccgtgtcacccacggtgatgaccccgtcccccgtcttcctcctctgatcttcggctaccgccacacctcccccgagtactggctcgatggcagcggtggtgacaagatcaactacaccatcaacgacatcaaggtctgcgagggtgctgccaacctgcagtgcaacggtggtaccctgggcctcgacattgctgctcacctgcactacttccaggccactggtgcctgcaacgccggtggtttcagctggcagcgctaccgctctgctgagagcgttgacaagcgtgccaccatgactgatgctgagctcgagaagaagctcaacagctacgtgcagatggacaaggagtacgtcaagaacaaccaggctcgctcctaaacgagacc

SEQ ID NO: 12 - DNA, 1057 bp, synthetic construct
  misc_feature 1..1057: note = Polynucleotide encoding synthetic polypeptide
  source 1..1057: mol_type = other DNA
ggtctcgaatgcttctcctctccctcctctccattgtcaccctcgctgttgcttctcctctgtccgttgaggagtacgccaaggccctcgaggagcgtgccgtcaccgtctcctcctccgagctcaacaacttcaagttctacatccagcacggtgctgctgcctactgcaactccgagactgctgctggtgccaacgtcacctgcactggcaacgcctgccccgagattgaggccaacggtgtcaccgttgttgcctccttcactggtaccaagactggtatcggtggctacgtctccaccgacaacaccaacaaggagatcgtcctttctttccgtggcagcaccaacatccgcaactggctgaccaacctggacttcggccaggatgactgctctctgacctccggctgcggtgtccactccggtttccagcgtgcctgggaggagattgccgacaacctgaccgctgctgttgccaaggccaagactgccaaccccgactacaaggttgttgccactggccactccctgggtggtgctgttgccaccctggctggtgccaacctccgtgctgctggtacccccctcgacatctacacctacggctctccccgtgtcggcaacgccgagcttgctgagttcatctccaaccagactggtggtgagttccgtgtcacccacggtgatgaccccgtcccccgtcttcctcctctgatcttcggctaccgccacacctcccccgagtactggctcgatggcagcggtggtgacaagatcaactacaccatcaacgacatcaaggtctgcgagggtgctgccaacctgcagtgcaacggtggtaccctgggcctcgacattgctgctcacctgcactacttccaggccactggtgcctgcaacgccggtggtttcagctgggctcgctaccgctctgctgagagcgttgacaagcgtgccaccatgactgatgctgagctcgagaagaagctcaacagctacgtgcagatggacaaggagtacgtcaagaacaaccaggctcgctcctaaacgagacc

SEQ ID NO: 13 - DNA, 1057 bp, synthetic construct
  misc_feature 1..1057: note = Polynucleotide encoding synthetic polypeptide
  source 1..1057: mol_type = other DNA
ggtctcgaatgcttctcctctccctcctctccattgtcaccctcgctgttgcttctcctctgtccgttgaggagtacgccaaggccctcgaggagcgtgccgtcaccgtctcctcctccgagctcaacaacttcaagttctacatccagcacggtgctgctgcctactgcaactccgagactgctgctggtgccaacgtcacctgcactggcaacgcctgccccgagattgaggccaacggtgtcaccgttgttgcctccttcactggtaccaagactggtatcggtggctacgtctccaccgacaacaccaacaaggagatcgtcctttctttccgtggcagcaccaacatccgcaactggctgaccaacctggacttcggccaggatgactgctctctgacctccggctgcggtgtccactccggtttccagcgtgcctgggaggagattgccgacaacctgaccgctgctgttgccaaggccaagactgccaaccccgactacaaggttgttgccactggccactccctgggtggtgctgttgccaccctggctggtgccaacctccgtgctgctggtacccccctcgacatctacacctacggctctccccgtgtcggcaacgccgagcttgctgagttcatctccaaccagactggtggtgagttccgtgtcacccacggtgatgaccccgtcccccgtcttcctcctctgatcttcggctaccgccacacctcccccgagtactggctcgatggcagcggtggtgacaagatcaactacaccatcaacgacatcaaggtctgcgagggtgctgccaacctgcagtgcaacggtggtaccctgggcctcgacattgctgctcacctgcactacttccaggccactggtgcctgcaacgccggtggtttcagctggcgccagtaccgctctgctgagagcgttgacaagcgtgccaccatgactgatgctgagctcgagaagaagctcaacagctacgtgcagatggacaaggagtacgtcaagaacaaccaggctcgctcctaaacgagacc

SEQ ID NO: 14 - DNA, 1057 bp, synthetic construct
  misc_feature 1..1057: note = Polynucleotide encoding synthetic polypeptide
  source 1..1057: mol_type = other DNA
ggtctcgaatgcttctcctctccctcctctccattgtcaccctcgctgttgcttctcctctgtccgttgaggagtacgccaaggccctcgaggagcgtgccgtcaccgtctcctcctccgagctcaacaacttcaagttctacatccagcacggtgctgctgcctactgcaactccgagactgctgctggtgccaacgtcacctgcactggcaacgcctgccccgagattgaggccaacggtgtcaccgttgttgcctccttcactggtaccaagactggtatcggtggctacgtctccaccgacaacaccaacaaggagatcgtcctttctttccgtggcagcaccaacatccgcaactggctgaccaacctggacttcggccaggatgactgctctctgacctccggctgcggtgtccactccggtttccagcgtgcctgggaggagattgccgacaacctgaccgctgctgttgccaaggccaagactgccaaccccgactacaaggttgttgccactggccactccctgggtggtgctgttgccaccctggctggtgccaacctccgtgctgctggtacccccctcgacatctacacctacggctctccccgtgtcggcaacgccgagcttgctgagttcatctccaaccagactggtggtgagttccgtgtcacccacggtgatgaccccgtcccccgtcttcctcctctgatcttcggctaccgccacacctcccccgagtactggctcgatggcagcggtggtgacaagatcaactacaccatcaacgacatcaaggtctgcgagggtgctgccaacctgcagtgcaacggtggtaccctgggcctcgacattgctgctcacctgcactacttccaggccactggtgcctgcaacgccggtggtttcagctggcgcgcctaccgctctgctgagagcgttgacaagcgtgccaccatgactgatgctgagctcgagaagaagctcaacagctacgtgcagatggacaaggagtacgtcaagaacaaccaggctcgctcctaaacgagacc

SEQ ID NO: 15 - DNA, 1033 bp, synthetic construct
  misc_feature 1..1033: note = Polynucleotide encoding synthetic polypeptide
  source 1..1033: mol_type = other DNA
ggtctcgaatgcttctcctctccctcctctccattgtcaccctcgctgttgcttctcctctgtccgttgaggagtacgccaaggccctcgaggagcgtgccgtcaccgtctcctcctccgagctcaacaacttcaagttctacatccagcacggtgctgctgcctactgcaactccgagactgctgctggtgccaacgtcacctgcactggcaacgcctgccccgagattgaggccaacggtgtcaccgttgttgcctccttcactggtaccaagactggtatcggtggctacgtctccaccgacaacaccaacaaggagatcgtcctttctttccgtggcagcaccaacatccgcaactggctgaccaacctggacttcggccaggatgactgctctctgacctccggctgcggtgtccactccggtttccagcgtgcctgggaggagattgccgacaacctgaccgctgctgttgccaaggccaagactgccaaccccgactacaaggttgttgccactggccactccctgggtggtgctgttgccaccctggctggtgccaacctccgtgctgctggtacccccctcgacatctacacctacggctctccccgtgtcggcaacgccgagcttgctgagttcatctccaaccagactggtggtgagttccgtgtcacccacggtgatgaccccgtcccccgtcttcctcctctgatcttcggctaccgccacacctcccccgagtactggctcgatggcagcggtggtgacaagatcaactacaccatcaacgacatcaaggtctgcgagggtgctgccaacctgcagtgcaacggtggtaccctgggcctcgacattgctgctcacctgcactacttccaggccactggtgcctgcaacgccggtggtttcagctggaaggagcgtgaggaagacatctccgatgctgagctcgagaagaagctcaacagctacgtgcagatggacaaggagtacgtcaagaacaaccaggctcgctcctaaacgagacc

SEQ ID NO: 16 - DNA, 1033 bp, synthetic construct
  misc_feature 1..1033: note = Polynucleotide encoding synthetic polypeptide
  source 1..1033: mol_type = other DNA
ggtctcgaatgcttctcctctccctcctctccattgtcaccctcgctgttgcttctcctctgtccgttgaggagtacgccaaggccctcgaggagcgtgccgtcaccgtctcctcctccgagctcaacaacttcaagttctacatccagcacggtgctgctgcctactgcaactccgagactgctgctggtgccaacgtcacctgcactggcaacgcctgccccgagattgaggccaacggtgtcaccgttgttgcctccttcactggtaccaagactggtatcggtggctacgtctccaccgacaacaccaacaaggagatcgtcctttctttccgtggcagcaccaacatccgcaactggctgaccaacctggacttcggccaggatgactgctctctgacctccggctgcggtgtccactccggtttccagcgtgcctgggaggagattgccgacaacctgaccgctgctgttgccaaggccaagactgccaaccccgactacaaggttgttgccactggccactccctgggtggtgctgttgccaccctggctggtgccaacctccgtgctgctggtacccccctcgacatctacacctacggctctccccgtgtcggcaacgccgagcttgctgagttcatctccaaccagactggtggtgagttccgtgtcacccacggtgatgaccccgtcccccgtcttcctcctctgatcttcggctaccgccacacctcccccgagtactggctcgatggcagcggtggtgacaagatcaactacaccatcaacgacatcaaggtctgcgagggtgctgccaacctgcagtgcaacggtggtaccctgggcctcgacattgctgctcacctgcactacttccaggccactggtgcctgcaacgccggtggtttcagctggcgccagcgtgaggaagagatctccgatgctgagctcgagaagaagctcaacagctacgtgcagatggacaaggagtacgtcaagaacaaccaggctcgctcctaaacgagacc

SEQ ID NO: 17 - DNA, 1024 bp, synthetic construct
  misc_feature 1..1024: note = Polynucleotide encoding synthetic polypeptide
  source 1..1024: mol_type = other DNA
ggtctcgaatgcttctcctctccctcctctccattgtcaccctcgctgttgcttctcctctgtccgttgaggagtacgccaaggccctcgaggagcgtgccgtcaccgtctcctcctccgagctcaacaacttcaagttctacatccagcacggtgctgctgcctactgcaactccgagactgctgctggtgccaacgtcacctgcactggcaacgcctgccccgagattgaggccaacggtgtcaccgttgttgcctccttcactggtaccaagactggtatcggtggctacgtctccaccgacaacaccaacaaggagatcgtcctttctttccgtggcagcaccaacatccgcaactggctgaccaacctggacttcggccaggatgactgctctctgacctccggctgcggtgtccactccggtttccagcgtgcctgggaggagattgccgacaacctgaccgctgctgttgccaaggccaagactgccaaccccgactacaaggttgttgccactggccactccctgggtggtgctgttgccaccctggctggtgccaacctccgtgctgctggtacccccctcgacatctacacctacggctctccccgtgtcggcaacgccgagcttgctgagttcatctccaaccagactggtggtgagttccgtgtcacccacggtgatgaccccgtcccccgtcttcctcctctgatcttcggctaccgccacacctcccccgagtactggctcgatggcagcggtggtgacaagatcaactacaccatcaacgacatcaaggtctgcgagggtgctgccaacctgcagtgcaacggtggtaccctgggcctcgacattgctgctcacctgcactacttccaggccactggtgcctgcaacgccggtggtttcagctggaagccccacatgtccgatgctgagctcgagaagaagctcaacagctacgtgcagatggacaaggagtacgtcaagaacaaccaggctcgctcctaaacgagacc

SEQ ID NO: 18 - DNA, 1033 bp, synthetic construct
  misc_feature 1..1033: note = Polynucleotide encoding synthetic polypeptide
  source 1..1033: mol_type = other DNA
ggtctcgaatgcttctcctctccctcctctccattgtcaccctcgctgttgcttctcctctgtccgttgaggagtacgccaaggccctcgaggagcgtgccgtcaccgtctcctcctccgagctcaacaacttcaagttctacatccagcacggtgctgctgcctactgcaactccgagactgctgctggtgccaacgtcacctgcactggcaacgcctgccccgagattgaggccaacggtgtcaccgttgttgcctccttcactggtaccaagactggtatcggtggctacgtctccaccgacaacaccaacaaggagatcgtcctttctttccgtggcagcaccaacatccgcaactggctgaccaacctggacttcggccaggatgactgctctctgacctccggctgcggtgtccactccggtttccagcgtgcctgggaggagattgccgacaacctgaccgctgctgttgccaaggccaagactgccaaccccgactacaaggttgttgccactggccactccctgggtggtgctgttgccaccctggctggtgccaacctccgtgctgctggtacccccctcgacatctacacctacggctctccccgtgtcggcaacgccgagcttgctgagttcatctccaaccagactggtggtgagttccgtgtcacccacggtgatgaccccgtcccccgtcttcctcctctgatcttcggctaccgccacacctcccccgagtactggctcgatggcagcggtggtgacaagatcaactacaccatcaacgacatcaaggtctgcgagggtgctgccaacctgcagtgcaacggtggtaccctgggcctcgacattgctgctcacctgcactacttccaggccactggtgcctgcaacgccggtggtttcagctggcgctctcgtcaggaagacatctccgatgctgagctcgagaagaagctcaacagctacgtgcagatggacaaggagtacgtcaagaacaaccaggctcgctcctaaacgagacc

SEQ ID NO: 19 - protein, 16 aa, synthetic construct
  REGION 1..16: note = Peptide motif
RRYRSAESVDKRATMT

SEQ ID NO: 20 - protein, 16 aa, synthetic construct
  REGION 1..16: note = Peptide motif
QRYRSAESVDKRATMT

SEQ ID NO: 21 - protein, 16 aa, synthetic construct
  REGION 1..16: note = Peptide motif
ARYRSAESVDKRATMT

SEQ ID NO: 22 - protein, 16 aa, synthetic construct
  REGION 1..16: note = Peptide motif
RQYRSAESVDKRATMT

SEQ ID NO: 23 - protein, 16 aa, synthetic construct
  REGION 1..16: note = Peptide motif
RAYRSAESVDKRATMT

SEQ ID NO: 24 - protein, 8 aa, synthetic construct
  REGION 1..8: note = Peptide motif
KEREEDIS

SEQ ID NO: 25 - protein, 8 aa, synthetic construct
  REGION 1..8: note = Peptide motif
RQREEEIS

SEQ ID NO: 26 - protein, 5 aa, synthetic construct
  REGION 1..5: note = Peptide motif
KPHMS

SEQ ID NO: 27 - protein, 8 aa, synthetic construct
  REGION 1..8: note = Peptide motif
RSRQEDIS

SEQ ID NO: 28 - protein, 346 aa, synthetic construct
  REGION 1..346: note = Synthetic polypeptide
MLLLPLLSAITLAVASPVALDDYVNSLEERAVGVTTTDFSNFKFYIQHGAAAYCNSEAAAGSKITCSNNGCPTVQGNGATIVTSFVGSKTGIGGYVATDSARKEIVVSFRGSTNIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATGACNAGGFSWRRYRSAESVDKRATMTDAELEKKLNSYVQMDKEYVKNNQARS

SEQ ID NO: 29 - protein, 338 aa, synthetic construct
  REGION 1..338: note = Synthetic polypeptide
MLLLPLLSAITLAVASPVALDDYVNSLEERAVGVTTTDFSNFKFYIQHGAAAYCNSEAAAGSKITCSNNGCPTVQGNGATIVTSFVGSKTGIGGYVATDSARKEIVVSFRGSTNIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATGACNAGGFSWKEREEDISDAELEKKLNSYVQMDKEYVKNNQARS

SEQ ID NO: 30 - protein, 335 aa, synthetic construct
  REGION 1..335: note = Synthetic polypeptide
MLLLPLLSAITLAVASPVALDDYVNSLEERAVGVTTTDFSNFKFYIQHGAAAYCNSEAAAGSKITCSNNGCPTVQGNGATIVTSFVGSKTGIGGYVATDSARKEIVVSFRGSTNIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATGACNAGGFSWKPHMSDAELEKKLNSYVQMDKEYVKNNQARS

SEQ ID NO: 31 - DNA, 1041 bp, synthetic construct
  misc_feature 1..1041: note = Polynucleotide encoding synthetic polypeptide
  source 1..1041: mol_type = other DNA
atgctcctccttcctctcctctccgccatcaccctggccgttgcctcccccgttgctctcgatgactacgtcaactccctcgaggagcgtgccgtcggtgtcaccaccaccgacttctccaacttcaagttctacatccagcacggtgctgctgcctactgcaactccgaggctgctgctggcagcaagatcacctgctccaacaacggctgccccaccgtccagggcaacggtgccaccattgtcacctccttcgtcggcagcaagactggtatcggtggctacgttgccaccgactctgctcgcaaggagattgtcgtctccttccgtggcagcaccaacatccgcaactggctgaccaacctggacttcggccaggaagactgctctcttgtctccggctgcggtgtccactccggtttccagcgtgcctggaacgagatctcctcccaggccactgctgccgttgcttctgctcgcaaggccaacccctccttcaacgtcatctccactggccactctcttggtggtgctgttgctgtccttgctgctgccaacctccgtgtcggtggtactcccgttgacatctacacctacggcagccctcgtgtcggcaacgcccagctgtctgctttcgtctccaaccaggctggtggtgaataccgtgtcacccacgccgatgaccccgttccccgtcttcctcctctgatcttcggttaccgccacaccactcccgagttctggctatccggtggtggtggtgacaaggtcgactacaccatctccgatgtcaaggtctgcgagggtgctgccaacctgggctgcaacggtggtaccctgggactcgacattgctgctcacctgcactacttccaggccactggtgcctgcaacgccggtggtttcagctggcgccgctaccgctctgctgagagcgttgacaagcgtgccaccatgactgatgctgagctcgagaagaagctcaacagctacgtgcagatggacaaggagtacgtcaagaacaaccaggctcgctcctaa

SEQ ID NO: 32 - DNA, 1017 bp, synthetic construct
  misc_feature 1..1017: note = Polynucleotide encoding synthetic polypeptide
  source 1..1017: mol_type = other DNA
atgctcctccttcctctcctctccgccatcaccctggccgttgcctcccccgttgctctcgatgactacgtcaactccctcgaggagcgtgccgtcggtgtcaccaccaccgacttctccaacttcaagttctacatccagcacggtgctgctgcctactgcaactccgaggctgctgctggcagcaagatcacctgctccaacaacggctgccccaccgtccagggcaacggtgccaccattgtcacctccttcgtcggcagcaagactggtatcggtggctacgttgccaccgactctgctcgcaaggagattgtcgtctccttccgtggcagcaccaacatccgcaactggctgaccaacctggacttcggccaggaagactgctctcttgtctccggctgcggtgtccactccggtttccagcgtgcctggaacgagatctcctcccaggccactgctgccgttgcttctgctcgcaaggccaacccctccttcaacgtcatctccactggccactctcttggtggtgctgttgctgtccttgctgctgccaacctccgtgtcggtggtactcccgttgacatctacacctacggcagccctcgtgtcggcaacgcccagctgtctgctttcgtctccaaccaggctggtggtgaataccgtgtcacccacgccgatgaccccgttccccgtcttcctcctctgatcttcggttaccgccacaccactcccgagttctggctatccggtggtggtggtgacaaggtcgactacaccatctccgatgtcaaggtctgcgagggtgctgccaacctgggctgcaacggtggtaccctgggactcgacattgctgctcacctgcactacttccaggccactggtgcctgcaacgccggtggtttcagctggaaggagcgtgaggaagacatctccgatgctgagctcgagaagaagctcaacagctacgtgcagatggacaaggagtacgtcaagaacaaccaggctcgctcctaa

SEQ ID NO: 33 - DNA, 1008 bp, synthetic construct
  misc_feature 1..1008: note = Polynucleotide encoding synthetic polypeptide
  source 1..1008: mol_type = other DNA
atgctcctccttcctctcctctccgccatcaccctggccgttgcctcccccgttgctctcgatgactacgtcaactccctcgaggagcgtgccgtcggtgtcaccaccaccgacttctccaacttcaagttctacatccagcacggtgctgctgcctactgcaactccgaggctgctgctggcagcaagatcacctgctccaacaacggctgccccaccgtccagggcaacggtgccaccattgtcacctccttcgtcggcagcaagactggtatcggtggctacgttgccaccgactctgctcgcaaggagattgtcgtctccttccgtggcagcaccaacatccgcaactggctgaccaacctggacttcggccaggaagactgctctcttgtctccggctgcggtgtccactccggtttccagcgtgcctggaacgagatctcctcccaggccactgctgccgttgcttctgctcgcaaggccaacccctccttcaacgtcatctccactggccactctcttggtggtgctgttgctgtccttgctgctgccaacctccgtgtcggtggtactcccgttgacatctacacctacggcagccctcgtgtcggcaacgcccagctgtctgctttcgtctccaaccaggctggtggtgaataccgtgtcacccacgccgatgaccccgttccccgtcttcctcctctgatcttcggttaccgccacaccactcccgagttctggctatccggtggtggtggtgacaaggtcgactacaccatctccgatgtcaaggtctgcgagggtgctgccaacctgggctgcaacggtggtaccctgggactcgacattgctgctcacctgcactacttccaggccactggtgcctgcaacgccggtggtttcagctggaagccccacatgtccgatgctgagctcgagaagaagctcaacagctacgtgcagatggacaaggagtacgtcaagaacaaccaggctcgctcctaa

SEQ ID NO: 34 - protein, 346 aa, synthetic construct
  REGION 1..346: note = Synthetic polypeptide
MLLLSLLSIVTLAVASPLSVEEYAKALEERAVTVSSSELNNFKFYIQHGAAAYCNSETAAGANVTCTGNACPEIEANGVTVVASFTGTKTGIGGYVSTDNTNKEIVLSFRGSINIRNWLTNLDFGQDDCSLTSGCGVHSGFQRAWEEIADNLTAAVAKAKTANPDYKVVATGHSLGGAVATLAGANLRAAGTPLDIYTYGSPRVGNAELAEFISNQTGGEFRVTHGDDPVPRLPPLIFGYRHTSPEYWLDGSGGDKINYTINDIKVCEGAANLQCNGGTLGLDIAAHLHYFQATDACNAGGFSWRRYRSAESVDKRATMTDAELEKKLNSYVQMDKEYVKNNQARS

(19) United States
(12) Patent Application Publication
(10) Pub. No.: US 2025/0304938 A1
(43) Pub. Date: Oct. 2, 2025

(54) LIPOLYTIC ENZYME VARIANTS

(71) Applicant: DSM IP Assets B.V., Maastricht (NL)

(72) Inventors: René Marcel DE JONG, ECHT (NL); Noël VAN PEIJ, ECHT (NL); Evert Tjeerd VAN RIJ, ECHT (NL)

(21) Appl. No.: 18/864,572

(22) PCT Filed: May 15, 2023
(86) PCT No.: PCT/EP2023/062995

§ 371 Date: Nov. 11, 2024

(30) Foreign Application Priority Data
May 16, 2022 (EP) 22173435.3

(51) Int. Cl. C12N 9/60; A21D 8/04

(52) U.S. Cl. CPC C12N 9/60; A21D 8/042; C12Y 304/21061

(57) ABSTRACT

The invention relates to the field of bakery ingredients. More specifically, the invention relates to a variant of a parent polypeptide, wherein the difference between the variant polypeptide and the parent polypeptide is that a KEX2 protease cleavage site which is present in the parent polypeptide, is absent in the variant polypeptide. The invention further relates to a process for preparing a dough wherein a variant polypeptide as disclosed herein is used and baked product prepared from the dough.

### FIELD

The invention relates to the field of bakery ingredients. More specifically, the invention relates to a variant of a parent polypeptide, wherein the difference between the variant polypeptide and the parent polypeptide is that a KEX2 protease cleavage site which is present in the parent polypeptide, is absent in the variant polypeptide. The invention further relates to a process for preparing a dough wherein a variant polypeptide as disclosed herein is used and a baked product prepared from the dough.

### BACKGROUND

In the baking industry, e.g. in industrial dough and bread production, processing aids are commonly used to improve properties of a dough and/or of a baked product. Dough properties that may be improved include stability, gas retaining capability, elasticity, extensibility, stickiness, machineability, moldability, properties of frozen dough, etcetera. Properties of a baked product that may be improved comprise loaf volume, crust crispiness, firmness, split, blistering, oven spring, crumb texture, crumb structure, crumb softness, flavour, relative staleness and shelf life.

It is known that dough made from wholemeal (also referred to as whole wheat) flour has a poorer stability than dough made from white flour. Consequently, at the end of proof wholemeal dough loses more leavening gas and the volume of the baked product of the wholemeal dough is lower as compared to the volume of a baked product made from white flour dough. In particular, during process handling when the dough is knocked or jarred, the dough volume is challenged and may partially collapse.

Cereal flour contains a certain amount of lipids and free fatty acids, and during storage of flour the amount of free fatty acids in the flour usually increases, for instance due to lipolysis of endogenous lipids. This is mostly noted during storage of wholemeal flour (see for instance Tait and Galliard, J Cereal Sci. 1988, 8:125-137 and Clayton and Morrison, Sci. Food Agric. 1972, 23, 721-735). The amount of free fatty acids in flour influences dough properties such as dough stability, and properties, taste and flavour of baked products made thereof.

Processing aids, such as chemical additives and enzymes are added to flour and/or dough to improve the properties of a dough or a baked product.

Chemical additives comprise emulsifiers, such as emulsifiers acting as dough conditioners such as diacetyl tartaric acid esters of mono/diglycerides (DATEM), sodium stearoyl lactylate (SSL), calcium stearoyl lactylate (CSL) or (distilled) mono- and diglycerides (MDG/DMG). Emulsifiers such as DATEM may also be used to increase or control the volume of a baked product. There is a growing demand of consumers to alternatives to chemical emulsifiers and therefore there is a need for non-chemical emulsifiers.

As alternative to chemical emulsifiers, lipolytic enzymes can be used that upon action on a substrate generate emulsifying molecules in situ. For instance, lipases are used to fully or partly replace DATEM. WO1998/026057 describes a phospholipase that can be used in a process for the production of bread. WO2009/106575 describes a lipolytic enzyme and its use in a process for making bread.

There is a continuing need for improved lipolytic enzymes that can be used as processing aids in the preparation of baked products, such as wholemeal baked products.

### FIGURES

FIG. 1. Depicted is a physical map of the pGBTOP-18 vector. The pGBTOP-18 vector is derived from the pGBTOP-16. Four Bsal sites were removed and two Bsal sites at 394 and 1090 were introduced to allow GoldenGate cloning.

### SEQUENCE LISTING

SEQ ID NO: 1 Reference polypeptide M15
SEQ ID NO: 2 Polynucleotide encoding reference polypeptide M15
SEQ ID NO: 3 Variant polypeptide M15_002
SEQ ID NO: 4 Variant polypeptide M15_004
SEQ ID NO: 5 Variant polypeptide M15_006
SEQ ID NO: 6 Variant polypeptide M15_008
SEQ ID NO: 7 Variant polypeptide M15_055
SEQ ID NO: 8 Variant polypeptide M15_056
SEQ ID NO: 9 Variant polypeptide M15_057
SEQ ID NO: 10 Variant polypeptide M15_066
SEQ ID NO: 11 Polynucleotide encoding reference polypeptide M15_002
SEQ ID NO: 12 Polynucleotide encoding variant polypeptide M15_004
SEQ ID NO: 13 Polynucleotide encoding variant polypeptide M15_006
SEQ ID NO: 14 Polynucleotide encoding variant polypeptide M15_008
SEQ ID NO: 15 Polynucleotide encoding variant polypeptide M15_055
SEQ ID NO: 16 Polynucleotide encoding variant polypeptide M15_056
SEQ ID NO: 17 Polynucleotide encoding variant polypeptide M15_057
SEQ ID NO: 18 Polynucleotide encoding variant polypeptide M15_066
SEQ ID NO: 19 Sequence of loop (linker) amino acids 305-320 of M15
SEQ ID NO: 20 Sequence of loop (linker) replacing amino acids 305-320 of M15
SEQ ID NO: 21 Sequence of loop (linker) replacing amino acids 305-320 of M15
SEQ ID NO: 22 Sequence of loop (linker) replacing amino acids 305-320 of M15
SEQ ID NO: 23 Sequence of loop (linker) replacing amino acids 305-320 of M15
SEQ ID NO: 24 Sequence of loop (linker) replacing amino acids 305-320 of M15
SEQ ID NO: 25 Sequence of loop (linker) replacing amino acids 305-320 of M15
SEQ ID NO: 26 Sequence of loop (linker) replacing amino acids 305-320 of M15
SEQ ID NO: 27 Sequence of loop (linker) replacing amino acids 305-320 of M15
SEQ ID NO: 28 Reference polypeptide LPV06
SEQ ID NO: 29 Variant polypeptide R3_072
SEQ ID NO: 30 Variant polypeptide R3_073
SEQ ID NO: 31 Polynucleotide encoding reference polypeptide LPV06
SEQ ID NO: 32 Polynucleotide encoding variant polypeptide R3_072
SEQ ID NO: 33 Polynucleotide encoding variant polypeptide R3_073
SEQ ID NO: 34: Parent polypeptide

### SUMMARY

Provided is a variant of a parent polypeptide, wherein the difference between the variant polypeptide and the parent polypeptide is that a KEX2 protease cleavage site which is present in the parent polypeptide, is absent in the variant polypeptide.

Further provided is a polynucleotide encoding the variant polypeptide.

Further provided is a recombinant host cell comprising the polynucleotide.

Further provided is a process for producing a variant polypeptide.

Further provided is a process for producing a mature polypeptide having lipolytic activity.

Further provided is a composition comprising the variant polypeptide.

Further provided is the use of the variant polypeptide or the composition in the production of a food product.

Further provided is a dough comprising the variant polypeptide.

Further provided is a process for the production of a baked product comprising baking the dough.

Further provided is a baked product.

### DEFINITIONS

The term ‘baked product’ refers to a baked food product prepared from a dough. Examples of baked products, whether of a white, brown, or wholegrain such as wholemeal or wholewheat type, include bread, typically in the form of loaves or rolls, French baguette-type bread, pastries, croissants, brioche, panettone, pasta, noodles (boiled or (stir-)fried), pita bread and other flat breads, tortillas, tacos, cakes, pancakes, muffins, cookies in particular biscuits, doughnuts, including yeasted doughnuts, bagels, pie crusts, steamed bread, crisp bread, brownies, sheet cakes, snack foods (e.g., pretzels, tortilla chips, fabricated snacks, fabricated potato crisps). Baked products are typically made by baking a dough at a suitable temperature for making the baked product such as a temperature between 100° C. and 300° C. A baked product as disclosed herein may be a wholemeal or a wholewheat bread.

The term “dough” is defined herein as a mixture of flour and other ingredients. Usually, dough is firm enough to knead or roll. The dough may be fresh, frozen, prepared or parbaked. Dough is usually made from basic dough ingredients including (cereal) flour, such as wheat flour or rice flour, water and optionally salt. For leavened products, primarily baker's yeast is used, and optionally chemical leavening compounds can be used, such as a combination of an acid (generating compound) and bicarbonate. Cereals from which flour can be made include maize, rice, wheat, barley, sorghum, millet, oats, rye, triticale, buckwheat, quinoa, spelt, einkorn, emmer, durum and kamut. The term dough herein also includes a batter. A batter is a semi-liquid mixture, being thin enough to drop or pour from a spoon, of one or more flours combined with liquids such as water, milk or eggs used to prepare various foods, including cake.

The term “pre-mix” is to be understood in its conventional meaning, i.e. as a mix of baking agents, generally including flour, starch, maltodextrin and/or salt, which may be used not only in industrial bread-baking plants/facilities, but also in retail bakeries. A pre-mix comprises a polypeptide having lipase activity as disclosed herein. A pre-mix may contain additives as mentioned herein.

Additives are in most cases added in powder form. Suitable additives include oxidants (including ascorbic acid, bromate and azodicarbonamide (ADA), reducing agents (including L-cysteine), emulsifiers (including without limitation mono- and diglycerides, monoglycerides such as glycerol monostearate (GMS), sodium stearoyl lactylate (SSL), calcium stearoyl lactylate (CSL), polyglycerol esters of fatty acids (PGE) and diacetyl tartaric acid esters of mono- and diglycerides (DATEM), propylene glycol monostearate (PGMS), lecithin), gums (including guar gum, pectin and xanthan gum), flavours, acids (including citric acid, propionic acid), starches, modified starches, humectants (including glycerol) and preservatives.

The term “control sequence” as used herein refers to components involved in the regulation of the expression of a coding sequence in a specific organism or in vitro. Examples of control sequences are transcription initiation sequences, termination sequences, promoters, leaders, signal peptides, propeptides, prepropeptides, or enhancer sequences; Shine-Delgarno sequences, repressor or activator sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion.

The term “expression” includes any step involved in the production of the polypeptide including, but not limited to, transcription, post transcriptional modification, translation, post-translational modification, and secretion.

An expression vector comprises a polynucleotide coding for a polypeptide, operably linked to the appropriate control sequences (such as a promoter, and transcriptional and translational stop signals) for expression and/or translation in vitro. The expression vector may be any vector (e.g., a plasmid or virus), which can be conveniently subjected to recombinant DNA procedures and can bring about the expression of the polynucleotide. The choice of the vector will typically depend on the compatibility of the vector with the cell into which the vector is to be introduced. The vectors may be linear or closed circular plasmids. The vector may be an autonomously replicating vector, i.e. a vector, which exists as an extra-chromosomal entity, the replication of which is independent of chromosomal replication, e.g., a plasmid, an extra-chromosomal element, a mini-chromosome, or an artificial chromosome. Alternatively, the vector may be one which, when introduced into the host cell, is integrated into the genome and replicated together with the chromosome(s) into which it has been integrated. The integrative cloning vector may integrate at random or at a predetermined target locus in the chromosomes of the host cell. The vector system may be a single vector or plasmid or two or more vectors or plasmids, which together contain the total DNA to be introduced into the genome of the host cell, or a transposon. Vectors preferred for use in bacteria are for example disclosed in WO2004/074468.

A host cell as defined herein is an organism suitable for genetic manipulation and one which may be cultured at cell densities useful for industrial production of a target product, such as a polypeptide according to the present invention. A host cell may be a host cell found in nature or a host cell derived from a parent host cell after genetic manipulation or classical mutagenesis. Advantageously, a host cell is a recombinant host cell. A host cell may be a prokaryotic, archaebacterial or eukaryotic host cell. A prokaryotic host cell may be, but is not limited to, a bacterial host cell. An eukaryotic host cell may be, but is not limited to, a yeast, a fungus, an amoeba, an algae, a plant, an animal, or an insect host cell.

A nucleic acid or polynucleotide sequence is defined herein as a nucleotide polymer comprising at least 5 nucleotide or nucleic acid units. A nucleotide or nucleic acid refers to RNA and DNA. The terms “nucleic acid” and “polynucleotide sequence” are used interchangeably herein. A nucleic acid or polynucleotide sequence is defined herein as a nucleotide polymer comprising at least 5 nucleotide or nucleic acid units. A nucleotide or nucleic acid refers to RNA and DNA.

The term “polypeptide” refers to a molecule comprising amino acid residues linked by peptide bonds and containing more than five amino acid residues. The term “protein” as used herein is synonymous with the term “polypeptide” and may also refer to two or more polypeptides. Thus, the terms “protein” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity in the presence of a specific substrate under certain conditions may be referred to as enzymes. It will be understood that, because of the degeneracy of the genetic code, a multitude of nucleotide sequences encoding a given polypeptide may be produced.

A polypeptide as disclosed herein may be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter(s) and terminator. The hybrid polypeptides may comprise a combination of partial or complete polypeptide sequences obtained from at least two different polypeptides wherein one or more may be heterologous to the host cell. Examples of fusion polypeptides and signal sequence fusions are for example as described in WO2010/121933.

The term “isolated polypeptide” as used herein means a polypeptide that is removed from at least one component, e.g. other polypeptide material, with which it is naturally associated. The isolated polypeptide may be free of any other impurities. The isolated polypeptide may be at least 50% pure, e.g., at least 60% pure, at least 70% pure, at least 75% pure, at least 80% pure, at least 85% pure, at least 80% pure, at least 90% pure, or at least 95% pure, 96%, 97%, 98%, 99%, 99.5%, 99.9% as determined by SDS-PAGE or any other analytical method suitable for this purpose and known to the person skilled in the art. An isolated polypeptide may be produced by a recombinant host cell.

A “mature polypeptide” is defined herein as a polypeptide in its final form and is obtained after translation of a mRNA into polypeptide and post-translational modifications of said polypeptide. Post-translational modifications include N-terminal processing, C-terminal truncation, glycosylation, phosphorylation and removal of leader sequences such as signal peptides, propeptides and/or prepropeptides by cleavage.

A “mature polypeptide coding sequence” means a polynucleotide that encodes a mature polypeptide.

The term “promoter” is defined herein as a DNA sequence that binds RNA polymerase and directs the polymerase to the correct downstream transcriptional start site of a nucleic acid sequence to initiate transcription. Suitable bacterial promotors are for instance disclosed in WO2004/074468.

The term “recombinant” when used with reference to a nucleic acid or protein indicates that the nucleic acid or protein has been modified in its sequence if compared to its native form by human intervention. The term “recombinant” when referring to a cell, such as a host cell, indicates that the genome of the cell has been modified in its sequence if compared to its native form by human intervention. The term “recombinant” is synonymous with “genetically modified”.

Sequence identity, or sequence homology are used interchangeable herein. To determine the percentage of sequence homology or sequence identity of two amino acid sequences or of two nucleic acid sequences, the sequences are aligned for optimal comparison purposes. To optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. Preferably, the alignment is performed over the full length of the sequences which are compared. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice,P. Longden,I. and Bleasby,A. Trends in Genetics 16, (6) pp276-277, http://emboss.bioinformatics.nl/). For protein sequences EBLOSUM62 is used for the substitution matrix. For nucleotide sequences, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms. After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest-identity”.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

A “synthetic molecule”, such as a synthetic nucleic acid or a synthetic polypeptide is produced by in vitro chemical or enzymatic synthesis. It includes, but is not limited to, variant nucleic acids made with optimal codon usage for host organisms of choice.

A synthetic nucleic acid may be optimized for codon use, preferably according to the methods described in WO2006/077258 and/or WO2008000632, which are herein incorporated by reference. WO2008/000632 addresses codon-pair optimization. Codon-pair optimization is a method wherein the nucleotide sequences encoding a polypeptide that have been modified with respect to their codon-usage, in particular the codon-pairs that are used, are optimized to obtain improved expression of the nucleotide sequence encoding the polypeptide and/or improved production of the encoded polypeptide. Codon pairs are defined as a set of two subsequent triplets (codons) in a coding sequence. Those skilled in the art will know that the codon usage needs to be adapted depending on the host species, possibly resulting in variants with significant homology deviation from SEQ ID NO: 2, but still encoding the polypeptide according to the invention.

As used herein, the terms “variant” or “mutant” can be used interchangeably. They can refer to either polypeptides or nucleic acids. Variants include substitutions, insertions, deletions, truncations, transversions, and/or inversions, at one or more locations relative to a reference sequence. Variants can be made for example by site-saturation mutagenesis, scanning mutagenesis, insertional mutagenesis, random mutagenesis, site-directed mutagenesis, and directed-evolution, as well as various other recombination approaches known to a skilled person in the art. Variant genes of nucleic acids may be synthesized artificially by known techniques in the art.

### DETAILED DESCRIPTION

Provided is a variant of a parent polypeptide, wherein the difference between the variant polypeptide and the parent polypeptide is that a KEX2 protease cleavage site which is present in the parent polypeptide, is absent in the variant polypeptide,
wherein the parent polypeptide has at least 80% sequence identity with the amino acid sequence as set forward in SEQ ID NO: 34
or
wherein the parent polypeptide has at least 80% sequence identity with the amino acid sequence as set forward in SEQ ID NO: 34 and wherein the variant polypeptide comprises at least one substitution of an amino acid residue at a position corresponding to any of the positions 113, 121, 138, 141, 179, 282, 284, 286, 295 of SEQ ID NO: 34.

The variant polypeptide is herein referred to as the variant polypeptide as disclosed herein, or as the variant polypeptide. The parent polypeptide is herein referred to as the parent polypeptide as disclosed herein, or as the parent polypeptide, or as the reference polypeptide. Absent herein means that the KEX2 protease cleavage site is functional in the parent polypeptide and is not functional or delayed (in time) functional (i.e. reduced functional) in the variant polypeptide; in view of the parent polypeptide, the KEX2 protease cleavage site in the variant polypeptide may not be present at all or may be present partially resulting in a non-functional or delayed (in time) functional (reduced functional) KEX2 protease cleavage site. Functional in the context of a KEX2 protease cleavage site means herein that the Kex2 protease cleavage site is recognized in the polypeptide by the KEX2 protease and that the polypeptide is cleaved by the KEX2 protease. Cleavage by a KEX2 protease is typically immediately after the KEX2 protease cleavage site. The term delayed (in time) functional (or reduced functional) means that cleavage is slower/delayed when compared to the parent polypeptide which comprises a (fully) functional KEX2 cleavage site. Most preferred, absent herein means that the KEX2 protease cleavage site is functional in the parent polypeptide and is delayed (in time) functional (reduced functional) in the variant polypeptide; compared to the parent polypeptide, the KEX2 protease cleavage site in the variant polypeptide may be present partially resulting in a delayed (in time) functional KEX2 protease cleavage site. Functional in the context of a KEX2 protease cleavage site means herein that the Kex2 protease cleavage site is recognized in the polypeptide by the KEX2 protease and that the polypeptide is cleaved by the KEX2 protease and is not delayed (in time). A typical functional KEX2 protease cleavage site is dibasic (RR, KK, KR or RK) in the parent polypeptide and a delayed (in time) functional (reduced functional) KEX2 protease cleavage site is mono basic (K or R on first or second position in the loop/linker sequence) in the variant polypeptide, i.e. it is most preferred that absent means a monobasic cleavage site with delayed (in time) proteolytic cleavage by kex2 protease upon secretion (compared to a dibasic cleavage site in the parent).

In the embodiments herein, the KEX2 protease cleaving site may be any KEX2 protease cleaving site known to the person skilled in the art. The KEX2 protease cleavage site may be a monobasic cleavage site or a dibasic cleavage site. The KEX2 protease cleaving site may comprise or consist of the amino acids KK, RR, RK or KR. The KEX2 protease cleaving site may comprise or consist of the amino acids RR. In the parent polypeptide, one or more KEX2 protease cleaving sites may be present. In an embodiment, one or more KEX2 protease cleaving sites is/are present in the parent polypeptide. In the variant polypeptide, one or more KEX2 protease cleaving sites may remain to be present. In an embodiment, no KEX2 protease cleaving site is present in the variant polypeptide. The term KEX2 protease cleavage site is herein interchangeably used with the terms KEX2 site, KEX2 cleavage site and KEX2 protease site.

As described above, the parent polypeptide has at least 80% sequence identity with the amino acid sequence as set forward in SEQ ID NO: 34
or
the parent polypeptide has at least 80% sequence identity with the amino acid sequence as set forward in SEQ ID NO: 34 and wherein the variant polypeptide further comprises at least one substitution of an amino acid residue at a position corresponding to any of the positions 113, 121, 138, 141, 179, 282, 284, 286, 295 of SEQ ID NO: 34.

The invention thus provides a variant of a parent polypeptide, wherein the difference between the variant polypeptide and the parent polypeptide is that a KEX2 protease cleavage site which is present in the parent polypeptide, is absent in the variant polypeptide, wherein preferably the KEX2 protease cleavage site consists of the amino acids KK, RR, RK or KR,
wherein the parent polypeptide has at least 80% sequence identity with the amino acid sequence as set forward in SEQ ID NO: 34.

Additionally, the invention provides a variant of a parent polypeptide, wherein the difference between the variant polypeptide and the parent polypeptide is that a KEX2 protease cleavage site which is present in the parent polypeptide, is absent in the variant polypeptide, wherein preferably the KEX2 protease cleavage site consists of the amino acids KK, RR, RK or KR,
wherein the parent polypeptide has at least 80% sequence identity with the amino acid sequence as set forward in SEQ ID NO: 34 and wherein the variant polypeptide comprises at least one substitution of an amino acid residue at a position corresponding to any of the positions 113, 121, 138, 141, 179, 282, 284, 286, 295 of SEQ ID NO: 34.

Reference herein to xx % (for example 80%) sequence identity needs to be understood as sequence identity, which is calculated over the full-length sequence, i.e. a full length sequence comparison.

In the embodiments herein, the KEX2 protease cleavage site may be present immediately before a pro-peptide within the parent polypeptide. In an embodiment, the pro-peptide having the KEX2 protease cleaving site immediately before it, is located at the C-terminus of the parent polypeptide.

In the embodiments herein, when the variant polypeptide is produced under the same circumstances as the parent polypeptide, the variant polypeptide has a higher yield compared to the parent polypeptide. Production of the variant and parent polypeptides may be performed according to any method known to the person skilled in the art. Preferably, the variant and parent polypeptides are produced according to the method as set forward in the examples herein. The yield may be at least 2% higher, such 3%, 4%, 5%, 6%, 7%, 8%, 9%, 10%, 15%, 20%, 30%, 40%, 50%, 60%, 70%, 80%, 90%, 100%, 200%, 300%, 400%, 500% or at least 1000% higher. The yield of a polypeptide may be defined as the amount of polypeptide produced. The person skilled in the art knows how to determine the yield of a polypeptide. Such can be determined by e.g. SDS PAGE, protein measurement and measurement of the absolute activity. In the embodiments herein, the parent and/or variant polypeptide may be a secreted polypeptide. Accordingly, when the variant polypeptide and the parent polypeptides are secreted polypeptides and the variant polypeptide is produced under the same circumstances as the parent polypeptide, the secreted variant polypeptide may have a higher yield as described herein above compared to the parent polypeptide.

In the embodiments herein, the variant polypeptide may be a fragment of the parent polypeptide wherein said variant polypeptide retains the functional features of the parent polypeptide. When the parent polypeptide is an enzyme, the variant polypeptide or the fragment retains the enzyme activity of the (mature) parent polypeptide.

In the embodiments herein, the variant polypeptide and the parent polypeptide may be enzymes, such as enzymes having lipolytic activity. Lipolytic activity is also referred to as a lipase activity. The variant polypeptide and the parent polypeptide as disclosed herein having lipase activity may have any suitable lipase activity, such as triacylglycerol lipase, galactolipase, and/or phospholipase activity. The variant polypeptide and the parent polypeptide as disclosed herein may have phospholipase A1 activity. Lipase activities may be determined according to known methods in the art. In an embodiment, the variant polypeptide and the parent polypeptide are EC 3.1.1.3 triacylglycerol lipases. An EC 3.1.1.3 triacylglycerol-lipase hydrolyses an ester bond in triglycerides (also known as triacylglycerol or triacyl glycerides (TAG)).

In the embodiments herein, the variant polypeptide, preferably the secreted variant polypeptide, may have at least 80%, 85%, 90%, 91%, 92%, 94%, 95%, 96%, 97%, 98%, at least 99% or 100% sequence identity to the sequence of amino acids 31 to 304 of SEQ ID NO: 34. In an embodiment, said variant polypeptide may comprise or consist of amino acids 34 to 304 of SEQ ID NO: 34, amino acids 34 to 302, amino acids 34 to 303, amino acids 31 to 302, amino acids 31 to 303, or amino acids 31 to 304. In an embodiment, the secreted variant polypeptide comprises or consists of amino acids 34 to 304 of SEQ ID NO: 34. The length of a polypeptide, such as the length of the variant polypeptide as disclosed herein may be determined by any method known to the person skilled in the art, such as LC-MS analysis. The variant polypeptide may be a biologically active fragment of SEQ ID NO: 34. Biologically active fragments herein include polypeptides which include fewer amino acids than the sequence of amino acids 31 to 304 of SEQ ID NO: 34 but which exhibit lipase activity of the polypeptide consisting of the sequence of amino acids 31 to 304 of SEQ ID NO: 34.

In the embodiments herein, the KEX2 protease cleavage site may be removed from the parent polypeptide to result in the variant polypeptide by any method known by the person skilled in the art. Preferably a method as set forward in the examples herein is used. In an embodiment, the KEX2 protease cleavage site is removed from the parent polypeptide to result in the variant polypeptide by:
(a) deletion or substitution of at least one amino acid residue of the KEX2 protease cleavage site, or
(b) replacement of the KEX2 protease cleavage site and optionally one or more contiguous amino acids adjacent to the KEX2 protease cleavage site by an amino acid motif not containing a KEX2 protease cleavage site. The one or more contiguous amino acids adjacent to the KEX2 protease cleavage site are preferably located immediately after protease cleavage site, so at the C-terminal part of the KEX2 protease cleavage site.

In the embodiments herein, the KEX2 protease cleavage site is preferably removed from the parent polypeptide to result in the variant polypeptide by replacing a linker motif adjacent to the C-terminal pro-peptide by an amino acid motif selected from the group consisting essentially or exactly of SEQ ID NO: 20, 21, 22, 23, 24, 25, 26 and 27. In an embodiment, the linker motif adjacent to the C-terminal pro-peptide in the parent polypeptide has essentially or exactly the amino acid sequence as set forward in SEQ ID NO: 19. In an embodiment, the linker motif adjacent to the C-terminal pro-peptide in the variant polypeptide has essentially or exactly the amino acid sequence as set forward in SEQ ID NO: 24. In a more preferred embodiment, the KEX2 protease cleavage site is preferably removed from the parent polypeptide to result in the variant polypeptide by replacing a linker motif adjacent to the C-terminal pro-peptide by an amino acid motif selected from the group consisting essentially or exactly of SEQ ID NO: 24, 25, 26 and 27.

In the embodiments herein, the parent polypeptide may have at least 80%, 85%, 90%, 95%, 96%, 97%, 98%, at least 99% or 100% sequence identity with the amino acid sequence as said forward in SEQ ID NO: 34.

In the embodiments herein, the variant polypeptide may comprise further amino acid substitutions. The variant polypeptide may comprise 1, 2, 3, 4, 5, 6, 7, 8, 9, 10, 11 or 12 or more further amino acid substitutions, deletions and/or insertions, whereby the polypeptide still has the same activity and/or function as the parent polypeptide. The variant polypeptide may comprise at least one substitution of an amino acid residue at a position corresponding to any of the positions 113, 121, 138, 141, 179, 282, 284, 286, 295 of SEQ ID NO: 34. The variant polypeptide may further comprise amino acid substitutions selected from the group consisting of: (D295S and V179M), (D295N and V179M), (D295S, I113H and V179M), (D295G and I113T), (D295N and I113N), (D295N, I113T, V179M, and N112D), (D295S, I113H, V179M, and I284M), (D295N and I113T), (D295S, I113T, V179M, and I284M), (D295S, I113T, V179M, N121D), (D295S, I113T and V179M), (D295S and I113N), and (D295N and I284T), wherein the substitutions are defined with reference to SEQ ID NO: 34.

In the embodiments herein, the variant polypeptide may comprise or consist of essentially or exactly the amino acid sequence selected from the group consisting of SEQ ID NO: 3, 4, 5, 6, 7, 8, 9 and 10 and may further comprise at least one substitution of an amino acid residue at a position corresponding to any of the positions 113, 121, 138, 141, 179, 282, 284, 286, 295.

In the embodiments herein, the variant polypeptide many comprise or consist of essentially or exactly the amino acid sequence selected from the group consisting of SEQ ID NO: 3, 4, 5, 6, 7, 8, 9 and 10 and may further comprise amino acid substitutions selected from the group consisting of: (D295S and V179M), (D295N and V179M), (D295S, I113H and V179M), (D295G and I113T), (D295N and I113N), (D295N, I113T, V179M, and N112D), (D295S, I113H, V179M, and I284M), (D295N and I113T), (D295S, I113T, V179M, and I284M), (D295S, I113T, V179M, N121D), (D295S, I113T and V179M), (D295S and I113N), and (D295N and I284T), wherein the substitutions are defined with reference to SEQ ID NO: 34.

Alternatively, the invention provides a variant of a parent polypeptide, wherein the difference between the variant polypeptide and the parent polypeptide is that a KEX2 protease cleavage site which is present in the parent polypeptide, is absent in the variant polypeptide, wherein preferably the KEX2 protease cleavage site consists of the amino acids KK, RR, RK or KR,
wherein the parent polypeptide has at least 80% sequence identity with the amino acid sequence as said forward in SEQ ID NO: 28 and
wherein the sequence as shown in SEQ ID NO: 19 is replaced by an amino acid motif selected from the group consisting of SEQ ID NO: 20, 21, 22, 24, 25, 26 and 27, preferably SEQ ID NO: 24, 25, 26 or 27.

In this alternative, the parent polypeptide may have at least 80%, 85%, 90%, 95%, 96%, 97%, 98%, at least 99% or 100% sequence identity with the amino acid sequence as said forward in SEQ ID NO: 28.

Further provided is a polynucleotide encoding the variant polypeptide as disclosed in the embodiments herein. In an embodiment, the polynucleotide is codon optimized or codon-pair optimized. Codon optimization is known to the person skilled in the art and any method known to the person skilled in the art may be used. Preferably, the method as set forward in the example herein is used. Said method is extensively described in WO2008/000632. Also provided is an expression vector comprising the polynucleotide as disclosed herein operably linked to at least one control sequence that directs expression of the polypeptide in a host cell. There are several ways of inserting a nucleic acid into a nucleic acid construct or an expression vector which are known to a person skilled in the art, see for instance Sambrook & Russell, Molecular Cloning: A Laboratory Manual, 3rd Ed., CSHL Press, Cold Spring Harbor, NY, 2001. It may be desirable to manipulate a nucleic acid encoding a polypeptide of the present invention with control sequences, such as promoter and terminator sequences. A variety of promoters can be used that can direct transcription in the host cells of the disclosure. A promoter sequence may be derived from a highly expressed gene. Strong constitutive promoters are well known and an appropriate one may be selected according to the specific sequence to be controlled in the host cell. Examples of suitable promotors are listed in WO 2009/106575, including examples of suitable promotors in filamentous fungi. All of the promoters mentioned therein are readily available in the art. Any terminator which is functional in a cell as disclosed herein may be used, which are known to a person skilled in the art. Examples of suitable terminator sequences in filamentous fungi include terminator sequences of a filamentous fungal gene, for example those listed in WO 2009/106575.

Further provided is a recombinant host cell comprising the polynucleotide as disclosed in the embodiments herein or comprising the expression vector as disclosed in the embodiments herein. A host cell may be a prokaryotic, archaebacterial or eukaryotic host cell. A prokaryotic host cell may be a bacterial host cell. A eukaryotic host cell may be a yeast, a fungus, an amoeba, an alga, a plant, an animal cell, such as a mammalian or an insect cell.

A eukaryotic cell may be a fungal cell, for example a yeast cell, such as a cell of the genus Candida, Hansenula, Kluyveromyces, Pichia, Saccharomyces, Schizosaccharomyces, or Yarrowia. A yeast cell may be from Kluyveromyces lactis, Saccharomyces cerevisiae, Hansenula polymorpha, Yarrowia lipolytica and Pichia pastoris, Candida krusei.

A eukaryotic cell may be a filamentous fungal cell. Several strains of filamentous fungi are readily accessible to the public in a number of culture collections, such as the American Type Culture Collection (ATCC), Deutsche Sammlung von Mikroorganismen und Zellkulturen GmbH (DSM), Westerdijk Fungal Biodiversity Institute formerly known as Centraalbureau Voor Schimmelcultures (CBS-KNAW), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia or the Fungal Genetics Stock Center (FGSC).

Preferred filamentous fungal cells belong to species of an Acremonium, Aspergillus, Chrysosporium, Myceliophthora, Penicillium, Talaromyces, Rasamsonia, Thielavia, Fusarium or Trichoderma genus, and for instance a species of Aspergillus niger, Aspergillus oryzae, Aspergillus awamori, Aspergillus foetidus, Aspergillus sojae, Aspergillus fumigatus, Acremonium alabamense Talaromyces emersonii, Rasamsonia emersonii, Chrysosporium lucknowense, Fusarium oxysporum, Myceliophthora thermophila, Trichoderma reesei, Thielavia terrestris or Penicillium chrysogenum. A filamentous fungal host cell belongs to the genus Aspergillus, for instance the species Aspergillus niger. Useful strains in the context of the disclosure may be Aspergillus niger CBS 513.88 (available with FGSC under ref. number A1513), CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255 (ATCC28089), Penicillium citrinum ATCC 38065, Penicillium chrysogenum P2, Thielavia terrestris NRRL8126, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila C1, Garg 27K, VKM-F 3500 D, Chrysosporium lucknowense C1, Garg 27K, VKM-F 3500 D, ATCC44006 and derivatives thereof.

A filamentous fungal cell may further comprise one or more modifications, preferably in its genome, such that the mutant filamentous fungal host cell is deficient in the cell in at least one product selected from glucoamylase (glaA), acid stable alpha-amylase (amyA), neutral alpha-amylase (amyBI and amyBII—WO2011009700), an α-1,3-glucan synthase (preferably AgsE or AgsE and AgsA, WO2014013074, WO2016066690), α-amylase AmyC (AmyC), a toxin, preferably ochratoxin and/or fumonisin (WO2011009700), a protease transcriptional regulator prtT (WO 00/20596, WO 01/68864, WO 2006/040312 and WO 2007/062936), PepA, a product encoded by the gene hdfA and/or hdfB (WO2005095624), Sec61 polypeptide (WO2005123763), a non-ribosomal peptide synthase npsE (WO2012001169), a zinc binuclear cluster transcriptional regulator-deficient strain (WO2018166943 and references therein) if compared to a parent host cell and measured under the same conditions.

In an embodiment, expression of the polynucleotide encoding the variant polypeptide is increased when the recombinant cell comprising the polynucleotide is cultivated under conditions conducive to the expression of the polynucleotide, when compared to expression of a polynucleotide encoding the reference polypeptide in an otherwise identical cell and both cells are cultivated under identical conditions. Preferably, the increased expression results in increased yield of the variant polypeptide, as described herein before. Preferably, the increased yield results in more protein produced as measured by for example a protein quantification method such as for example Bradford, Biuret, Lowry, BCA, HPLC, UV-VIS (280 nm).

Preferably, the increased yield results in more active protein produced as measured by for example a lipase enzyme analysis method suitable for the lipase enzyme produced and which are listed in for example WO2018114912 or WO2018114938.

Further provided is a process for producing a variant polypeptide as disclosed herein comprising cultivating the host cell as disclosed herein under conditions conducive to production of the variant polypeptide and optionally recovering the variant polypeptide. The person skilled in the art knows how to perform a process for preparing a polypeptide having lipase activity depending on the host cell used. A suitable fermentation medium usually comprises a carbon and nitrogen source. Usually, a fermentation medium has a pH value of between 3 and 8. A suitable temperature at which a host cell is cultivated is usually between 25 and 60° C.

Host cells can be cultivated in shake flasks, or in fermenters having a volume of 0.5 or 1 litre or larger up to 10 to 100 or more cubic metres. Cultivation may be performed aerobically or anaerobically depending on the requirements of a host cell.

The variant polypeptide as disclosed herein may be recovered or isolated from the fermentation medium. Recovering or isolating a polypeptide from a fermentation medium may for instance be performed by centrifugation, filtration, and/or ultrafiltration.

Also provided is a process for producing a polypeptide having lipolytic activity comprising cultivating the host cell as disclosed herein under conditions conducive to production of a variant polypeptide, recovering the variant polypeptide and activating the variant polypeptide to obtain said mature polypeptide having lipolytic activity. Preferably, the polypeptide having lipolytic activity has at least 80% identity to the mature polypeptide of SEQ ID NO: 1, SEQ ID NO: 34, SEQ ID NO:28 or SEQ ID NO: 34 which further comprises at least one substitution of an amino acid residue at a position corresponding to any of the positions 113, 121, 138, 141, 179, 282, 284, 286, 295 of SEQ ID NO: 34. Alternatively phrased the polypeptide having lipolytic activity has at least 80% identity to amino acids 31 to 304 of SEQ ID NO: 1, SEQ ID NO: 34, SEQ ID NO: 28 or SEQ ID NO: 34 which further comprises at least one substitution of an amino acid residue at a position corresponding to any of the positions 113, 121, 138, 141, 179, 282, 284, 286, 295 of SEQ ID NO: 34 (i.e. the polypeptide having lipolytic activity does not comprise a signal sequence, N-terminal propeptide and C-terminal propeptide). Activation of the variant polypeptide is for example obtained as described in the examples herein: pH treatment or diacetate treatment. The aim of the pH treatment is to obtain a pH around pH 5 such as pH 5.0±0.5 at 30° C., preferably pH 5.0±0.3 at 30° C. or more preferably pH 5.0±0.1 at 30° C. A suitable final diacetate concentration is a concentration of least 15 g/kg broth. The activation results in the production of the mature polypeptide having lipolytic activity.

Further provided is a process of producing a variant polypeptide as disclosed herein, which process comprises:
a) providing a parent polypeptide comprising a KEX2 protease cleavage site,
b) removing the KEX2 protease cleavage site from the parent polypeptide,
c) optionally, substituting one or more further amino acids in the parent polypeptide,
d) preparing the variant polypeptide resulting from steps a) to c),
e) optionally, recovering the variant polypeptide.

The person skilled in the art knows how to remove an amino acid sequence from a parent polypeptide. Preferably, a method as set forward in the examples herein is used.

Removal may include substitutions, insertions, deletions, truncations, transversions, and/or inversions, at the KEX protease cleaving site. Such modifications can be made for example by site-saturation mutagenesis, scanning mutagenesis, insertional mutagenesis, random mutagenesis, site-directed mutagenesis, and directed-evolution, as well as various other recombination approaches known to the skilled person in the art. Variant polynucleotides may be synthesized artificially by known techniques in the art. Such process may include expressing a gene encoding the variant polypeptide in a suitable recombinant host cell and cultivating the host cell to generate the variant polypeptide.

Preferably, step b) comprises substituting the KEX2 protease cleavage site from the parent polypeptide by substituting SEQ ID NO: 19 by SEQ ID NO: 24, 25, 26 or 27. As disclosed in the experimental part herein, such substitutions result in surprisingly high expression levels.

Preferred is, a process of producing a variant polypeptide as disclosed herein, which process comprises:
a) providing a parent polypeptide comprising a KEX2 protease cleavage site,
b) removing the KEX2 protease cleavage site from the parent polypeptide,
c) optionally, substituting one or more further amino acids in the parent polypeptide,
d) preparing the variant polypeptide resulting from steps a) to c),
e) optionally, recovering the variant polypeptide.
wherein the parent polypeptide has at least 80% sequence identity with the amino acid sequence as said forward in SEQ ID NO: 34
or
wherein the parent polypeptide has at least 80% sequence identity with the amino acid sequence as said forward in SEQ ID NO: 34 and wherein the variant polypeptide comprises at least one substitution of an amino acid residue at a position corresponding to any of the positions 113, 121, 138, 141, 179, 282, 284, 286, 295 of SEQ ID NO: 34
or
wherein the parent polypeptide has at least 80% sequence identity with the amino acid sequence as said forward in SEQ ID NO: 28
or
wherein the parent polypeptide has at least 80% sequence identity with the amino acid sequence as said forward in SEQ ID NO: 1

In the embodiments herein, the KEX2 protease cleavage site is preferably removed (in step b of the above method) from the parent polypeptide to result in the variant polypeptide by replacing a linker motif adjacent to the C-terminal pro-peptide by an amino acid motif selected from the group consisting essentially or exactly of SEQ ID NO: 20, 21, 22, 23, 24, 25, 26 and 27. In an embodiment, the linker motif adjacent to the C-terminal pro-peptide in the parent polypeptide has essentially or exactly the amino acid sequence as set forward in SEQ ID NO: 19. In an embodiment, the linker motif adjacent to the C-terminal pro-peptide in the variant polypeptide has essentially or exactly the amino acid sequence as set forward in SEQ ID NO: 24, 25, 26 or 27.

Alternatively is provided, a process of producing a variant polypeptide as disclosed herein, which process comprises:
a) providing a parent polypeptide comprising a KEX2 protease cleavage site,
b) removing the KEX2 protease cleavage site from the parent polypeptide,
c) optionally, substituting one or more further amino acids in the parent polypeptide,
d) preparing the variant polypeptide resulting from steps a) to c),
e) optionally, recovering the variant polypeptide.
   wherein the parent polypeptide has at least 80% sequence identity with the amino acid sequence as said forward in SEQ ID NO: 28 and
wherein the sequence as shown in SEQ ID NO: 19 is replaced by an amino acid motif selected from the group consisting of SEQ ID NO: 20, 21, 22, 24, 25, 26 and 27, preferably SEQ ID NO: 24, 25, 26 or 27, most preferably SEQ ID NO: 24 or 26.

Further provided is a composition comprising the variant polypeptide as disclosed in the embodiments herein and further comprising one or more compounds selected from the group consisting of milk powder, gluten, granulated fat, an additional enzyme, an amino acid, a salt, an oxidant, a reducing agent, an emulsifier, sodium stearoyl lactylate, calcium stearoyl lactylate, polyglycerol esters of fatty acids and diacetyl tartaric acid esters of mono- and diglycerides, a gum, a flavour, an acid, a starch, a modified starch, a humectant and a preservative. A composition as disclosed herein may be a solid or fluid composition. A composition as disclosed herein may comprise one or more compounds selected from the group consisting of: milk powder, gluten, granulated fat, an additional enzyme, an amino acid, a salt, an oxidant, a reducing agent, an emulsifier, sodium stearoyl lactylate, calcium stearoyl lactylate, polyglycerol esters of fatty acids and diacetyl tartaric acid esters of mono- and diglycerides, a gum, a flavour, an acid, a starch, a modified starch, a humectant and a preservative. The term composition includes a pre-mix. A composition as disclosed herein may comprise one or more further enzyme(s) such as an amylase such as an alpha-amylase, for example a fungal alpha-amylase (which may be useful for providing sugars fermentable by yeast), a beta-amylase; a glucanotransferase; a peptidase in particular, an exopeptidase (which may be useful in flavour enhancement); a transglutaminase; a cellulase; a hemicellulase, in particular a pentosanase such as xylanase (which may be useful for the partial hydrolysis of pentosans, more specifically arabinoxylan, which increases the extensibility of the dough); protease (which may be useful for gluten weakening in particular when using hard wheat flour); a protein disulfide isomerase, e.g., a protein disulfide isomerase as disclosed in WO 95/00636; a glycosyltransferase; a peroxidase (which may be useful for improving the dough consistency); a laccase; an oxidase, such as an hexose oxidase, a glucose oxidase, aldose oxidase, pyranose oxidase; a lipoxygenase; L-amino acid oxidase (which may be useful in improving dough consistency) and/or an asparaginase.

Further provided is the use of the variant polypeptide as disclosed herein or of the composition as disclosed herein for the production of a food product, preferably in the production of a dough and/or a baked product. Such use will typically comprise adding the variant polypeptide as disclosed herein or of the composition as disclosed herein to a dough. In an embodiment, the use comprises replacing at least part of a chemical emulsifier in the production of a dough and/or a baked product. At least part may be at least 10%, 20%, 30%, 40%, 50%, 60%, 70%, 80%, 90%, 95%, 96%, 97%, 98%, at least 99%, or 100%.

Further provided is a dough comprising the variant polypeptide as disclosed herein, a variant polypeptide obtainable by process of producing a variant polypeptide as disclosed herein or the composition as disclosed herein. A dough or a baked product as disclosed in the embodiments herein may comprise any suitable cereal flour, for instance wholemeal flour, or a mixture of different flours. Cereal flour may also comprise bran, grains and/or seeds. Cereals include maize, rice, wheat, barley, sorghum, millet, oats, rye, triticale, buckwheat, quinoa, spelt, einkorn, emmer, durum and kamut. Wholemeal flour, also referred to as wholewheat flour, is flour made from the entire wheat kernel or grain including the outer part. Flour may comprise a free fatty acid content of between 0.01 to 0.8 w/w %, for instance 0.05 to 0.6 w/w %, for instance a free fatty acid content of between 0.1 to 0.5 w/w % or between 0.14 to 0.4 w/w %. During storage the content of free fatty acids in flour usually increases. Free fatty acids may for instance be linoleic acid (C18:2), palmitic acid (C16:0), oleic acid (C18:1), linolenic acid (C18:3) (see for instance MacMurray and Morrison, J.Sci. Food Agr. 21:520-528 (1970). Free fatty acids in flour may be determined by methods known to a person skilled in the art, for instance as disclosed in Fierens et al, J. of Cereal Science 65 (2015), p. 81-87.

Further provided is a process for the production of a dough comprising a step of combining an effective amount of the variant polypeptide as disclosed herein, an effective amount of the variant polypeptide obtainable by the process as disclosed herein or an effective amount of the composition as disclosed herein with at least one dough ingredient, such as the ingredients set forward here above.

Further provided is a process for the production of a baked product, which process comprises baking a dough as disclosed herein. Also provided is a baked product obtainable by a process as disclosed herein. In the embodiments herein, the baked product may be a bread, a cake or a baked product prepared from a laminated dough. The baked product may have at least one improved property selected from the group consisting of increased volume, improved flavour, improved crumb structure, improved crumb softness, improved crispiness, reduced blistering and improved anti-staling.

In the embodiments herein, the dough may comprise a wholemeal flour and/or may comprise a flour comprising a free fatty acid content of between 0.01 to 0.8 w/w %, such as 0.05 to 0.6 w/w %, for instance 0.1 w/w % to 0.5 w/w % or 0.2 to 4 w/w % free fatty acids. Fatty acids in grains or cereals are known and comprise palmitic acid, oleic acid, linoleic acid and/or linolenic acid. Flour may be a flour that has been stored, for instance stored for 1 day to 10 years, for instance for 1 month to 5 years, or stored for 2 months to 1 year.

### Further Embodiments of the Invention

1. A variant of a parent polypeptide, wherein the difference between the variant polypeptide and the parent polypeptide is that a KEX2 protease cleavage site which is present in the parent polypeptide, is absent in the variant polypeptide.

2. A variant polypeptide according to embodiment 1, wherein the KEX2 protease cleavage site consists of the amino acids KK, RR, RK or KR.

3. A variant polypeptide according to embodiment 1 or 2, wherein the KEX2 protease cleavage site is present immediately before a pro-peptide within the parent polypeptide and wherein, preferably, the pro-peptide having the KEX2 protease cleavage site immediately before it, is located at the C-terminus of the parent polypeptide.

4. A variant polypeptide according to any one of the preceding embodiments, wherein, when produced under the same circumstances as the parent polypeptide, the variant polypeptide has a higher yield compared to the parent polypeptide.

5. A variant polypeptide according to any one of the preceding embodiments, wherein the variant polypeptide and parent polypeptide have lipolytic activity and are preferably EC 3.1.1.3 triacylglycerol lipases.

6. A variant polypeptide according to any one of the preceding embodiments, wherein the KEX2 protease cleavage site is removed by:
(a) deletion or substitution of at least one amino acid residue of the KEX2 protease cleavage site, or
(b) replacement of the KEX2 protease cleavage site and optionally one or more contiguous amino acids adjacent to the KEX2 protease cleavage site by an amino acid motif not containing a KEX2 protease cleavage site.

7. A variant polypeptide according to embodiment 6, wherein the linker motif adjacent to the C-terminal pro-peptide is replaced by an amino acid motif selected from the group consisting of SEQ ID NO: 20, 21, 22, 23, 24, 25, 26 and 27.

8. A variant polypeptide according to embodiment 7, wherein the linker motif adjacent to the C-terminal pro-peptide in the parent polypeptide has the amino acid sequence as said forward in SEQ ID NO: 19.

9. A variant polypeptide according to any one of the preceding embodiments, wherein the parent polypeptide has at least 60% sequence identity with the amino acid sequence as said forward in SEQ ID NO: 34.

10. A variant polypeptide according to any one of the preceding embodiments, wherein the variant polypeptide comprises at least one substitution of an amino acid residue at a position corresponding to any of the positions 113, 121, 138, 141, 179, 282, 284, 286, 295 of SEQ ID NO: 34.

11. A variant polypeptide according to any one of the preceding embodiments, wherein the variant polypeptide has the amino acid sequence selected from the group consisting of SEQ ID NO: 3, 4, 5, 6, 7, 8, 9 and 10 and preferably comprises at least one substitution of an amino acid residue at a position corresponding to any of the positions 113, 121, 138, 141, 179, 282, 284, 286, 295.

12. A polynucleotide encoding the variant polypeptide according to any one of embodiments 1 to 11, wherein the polynucleotide preferably is codon optimized.

13. A recombinant host cell comprising the polynucleotide according to embodiment 12.

14. A process for producing a variant polypeptide according to any one of embodiments 1 to 11 comprising cultivating the host cell of embodiment 13 under conditions conducive to production of the variant polypeptide and recovering the variant polypeptide.

15. A process of producing a variant polypeptide, which process comprises:
a) providing a parent polypeptide comprising a KEX2 protease cleavage site,
b) removing the KEX2 protease cleavage site from the parent polypeptide,
c) optionally, substituting one or more further amino acids in the parent polypeptide,
d) preparing the variant polypeptide resulting from steps a) to c),
e) optionally, recovering the variant polypeptide.

16. A composition comprising the variant polypeptide according to any one of the embodiments 1 to 11 or obtainable by the method according to embodiment 15, and further comprising one or more compounds selected from the group consisting of milk powder, gluten, granulated fat, an additional enzyme, an amino acid, a salt, an oxidant, a reducing agent, an emulsifier, sodium stearoyl lactylate, calcium stearoyl lactylate, polyglycerol esters of fatty acids and diacetyl tartaric acid esters of mono- and diglycerides, a gum, a flavour, an acid, a starch, a modified starch, a humectant and a preservative.

17. Use of the variant polypeptide according to any one of embodiments 1 to 11 or of the composition according to embodiment 16 in the production of a food product, preferably in the production of a dough and/or a baked product.

18. The use according to embodiment 17, wherein the use comprises replacing at least part of a chemical emulsifier in the production of a dough and/or a baked product.

19. A dough comprising the variant polypeptide according to any one of the embodiments 1 to 11, a variant polypeptide obtainable by the method according to embodiment 15 or the composition according to embodiment 16.

20. A process for the production of a dough comprising a step of combining an effective amount of the variant polypeptide according to any one of the embodiments 1 to 11, an effective amount of the variant polypeptide obtainable by the process according to embodiment 15 or an effective amount of the composition according to embodiment 16 with at least one dough ingredient.

21. A process for the production of a baked product, which process comprises baking the dough according to embodiment 19 or the dough obtained by the process of embodiment 20.

22. A baked product obtainable by the process according to embodiment 21.

23. A baked product according to embodiment 22, wherein the product is a bread, a cake or baked product prepared from a laminated dough.

24. A baked product according to embodiment 22 or 23 having at least one improved property selected from the group consisting of increased volume, improved flavour, improved crumb structure, improved crumb softness, improved crispiness, reduced blistering and improved anti-staling.

25. Use according to embodiment 17 or 18, a dough according to embodiment 19, a process according to embodiment 20 or 21, or a baked product according to embodiment 22, 23 or 24, wherein the dough comprises a wholemeal flour and/or a flour comprising a free fatty acid content of between 0.01 to 0.8 w/w %.

### EXAMPLES

Standard genetic techniques, such as overexpression of enzymes in the host cells, genetic modification of host cells, or hybridisation techniques, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation, genetic modification etc of fungal host cells are known from e.g. EP0635574, WO98/46772, WO99/60102 and WO00/37671, WO90/14423, EP0481008, EP0635574 and U.S. Pat. No. 6,265,186.

### Materials and Methods

### Strains

WT 1: This Aspergillus niger strain is used as a wild-type strain. This strain is deposited at the CBS Institute under the deposit number CBS 513.88.

GBA 306: The construction of GBA 306 using WT1 as starting strain has been described in detail in WO2011/009700. This GBA 306 strain has the following genotype: ΔglaA, ΔpepA, ΔhdfA, an adapted BamHI amplicon, ΔamyBII, ΔamyBI, and ΔamyA.

### Vectors

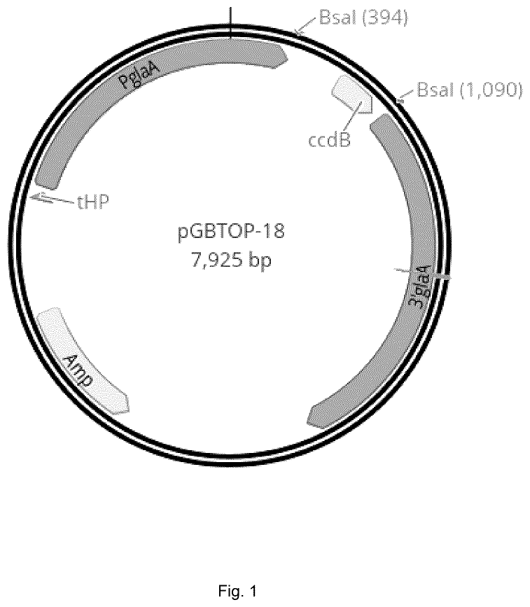

The pGBTOP-16 vector described in WO15177171 A1, WO16097270 A1, WO16/193292 A1 was modified to allow Golden Gate cloning (New England Biolabs). The four Bsal sites present in pGBTOP-16 were removed and two Bsal sites were introduced to allow cloning, one at the 3′-end of PglaA (promoter) fragment and one at the 5′-end of the 3′glaA (terminator) fragment. This resulted in vector pGBTOP-18 (FIG. 1).

### Production of the Lipases of the Invention in Shake Flask

The lipolytic enzymes were produced in shake flask by growing the A. niger strains as indicated. Inoculation of spores was done in a pre-culture of CSL-medium and subsequently CSM-medium culture for 4 days at 34° C. as described in detail in Example 1 of WO2009/106575, to generate broth samples and (cell-free) supernatant samples for subsequent analyses.

### Protein Content (Bradford/SDS-PAGE)

Protein content is determined using the Bradford assay (Bradford, M. M. (1976), “Rapid and sensitive method for the quantitation of microgram quantities of protein utilizing the principle of protein-dye binding”, Anal. Biochem. 72:248-254) in combination with an SDS-PAGE verification to confirm that samples show >80% purity based on band intensity.

pH adjustment

The pH adjustment of phospholipase samples was performed after shake flask fermentation at end of fermentation both broth samples as well as supernatant samples by incubation of a sample at temperature, pH and time as indicated. For pH adjustment, the pH of 50 g of the broth or 50 g of supernatant (obtained after 10 min centrifugation and taking the upper liquid) obtained from the flasks was adjusted with 4 N NaOH or 3.5 M phosphoric acid to a setpoint of pH 5.0±0.1 at 30° C. Phospholipase activities were corrected/normalized for the dilution factor, as a result of pH adjustment with alkaline/acid.

### Diacetate Treatment

For diacetate treatment, samples with roughly 50 g broth or 50 gr supernatant (vide super. “pH Treatent”), 350 g/kg sodium diacetate stock solutions were added to 15 g/kg final sodium diacetate concentration. Diacetate treatment was tested at pH 5.0±0.1 at 30° C. for the incubation times as indicated. Phospholipase activities were corrected/normalized for the dilution factor as a result of diacetate solution addition and/or pH adjustment with alkaline/acid.

### Phospholipase Enzymatic Activity Assay (PLE)

The following solutions were used to measure PLE Phospholipase enzymatic activity:
Substrate solution of 0.7% (w/v) L-α-phosphatidylcholine from egg yolk (Sigma 61755, Zwijndrecht, the Netherlands) in 60 mM acetate buffer pH 5.5 also containing 1.7% triton X-100
Reagent R1 and R2 prepared by following the instructions described in the package insert of the Wako HR series NEFA-HR (2) diagnostic kit (FUJIFILM/Wako Chemicals).

Activity analysis was executed with a programmed Konelab analyzer (Thermo-Fisher) as also detailed in Examples section of WO18114912 (Lipolytic Enzyme Variants). The reaction sequence was started by equilibrating 40 μL substrate solution for 2 minutes to reach 37° C. Subsequently 10 μL enzyme solution with activity between 0.015-0.075 U/mL was added and incubated for 20 minutes at 37° C. Then, for determining the free fatty acid content, 130 μL NEFA-R1 reagent was added and incubated for 2 minutes followed by addition of 65 μL NEFA-R2 reagent and 3 minutes of incubation. At the end of incubation, the absorbance was measured at 540 nm. Blanks were measured by changing the reaction sequence as follows: 130 μL NEFA-R1 reagent was equilibrated for 65 seconds at 37° C., then incubated with 10 μL enzyme solution for 65 seconds, followed by 2 minutes incubation with 40 μL substrate and 3 minutes incubation with 65 μL NEFA-R2 reagent. At the end of incubation, the absorbance was measured at 540 nm. A 1.0 mM oleic acid standard (NEFA standard 270-77000/FUJIFILM/Wako Chemicals) was also analyzed in the same way for determining the response factor.

Activity was calculated as follows:

$$U / mL = \frac{Δ OD \times df}{RF \times t}$$

ΔOD=ODₛₐₘₚₗₑ−OD_{blank}
RF=Response Factor (mL/μmol)
t=incubation time (20 minutes)
df=dilution factor of sample

1 U PLE is defined as the amount of enzyme that liberates one micromole of free fatty acid per minute under the conditions of the test (pH 5.5/37° C.).

### Example 1: Cloning and Expression of a Lipolytic Enzyme Variant

### Example 1.1. Cloning and Expression

The protein sequence (amino acid sequence) of the reference polypeptide (also referred to as M15) SEQ ID NO: 1. This polypeptide sequence is comprised of a signal peptide 1-15 (bold), a N-terminal pro-peptide 16-30 (underlined), mature polypeptide 31-304 having lipolytic activity (plain), C-terminal pro-peptide 307-346 (underlined); the Kex2 protease cleavage site is bold underlined.

(346 amino acids)
SEQ ID NO: 1
MLLLSLLSIVTLAVASPLSVEEYAKALEERAVTVSSSELNNFKFYIQHG
AAAYCNSETAAGANVTCTGNACPEIEANGVTVVASFTGTKTGIGGYVST
DNTNKEIVLSFRGSTNIRNWLTNLDFGQDDCSLTSGCGVHSGFQRAWEE
IADNLTAAVAKAKTANPDYKVVATGHSLGGAVATLAGANLRAAGTPLDI
YTYGSPRVGNAELAEFISNQTGGEFRVTHGDDPVPRLPPLIFGYRHTSP
EYWLDGSGGDKINYTINDIKVCEGAANLQCNGGTLGLDIAAHLHYFQAT
GACNAGGFSWRRYRSAESVDKRATMTDAELEKKLNSYVQMDKEYVKNNQ
ARS

A codon-adapted DNA sequence for expression of the lipolytic enzyme proteins (lipolytic enzyme variants and reference polypeptide) in Aspergillus niger was designed containing additional Bsal type II restriction enzyme sites (bold) to enable subcloning in the Aspergillus expression vector pGBTOP-18. Codon adaptation was performed as described in WO2008/000632. The codon optimized DNA sequence for expression of the gene encoding the reference polypeptide of SEQ ID NO: 1 in A. niger is shown in SEQ ID NO: 2.

SEQ ID NO: 2
GGTCTCGAATGCTTCTCCTCTCCCTCCTCTCCATTGTCACCCTCGCTGT
TGCTTCTCCTCTGTCCGTTGAGGAGTACGCCAAGGCCCTCGAGGAGCGT
GCCGTCACCGTCTCCTCCTCCGAGCTCAACAACTTCAAGTTCTACATCC
AGCACGGTGCTGCTGCCTACTGCAACTCCGAGACTGCTGCTGGTGCCAA
CGTCACCTGCACTGGCAACGCCTGCCCCGAGATTGAGGCCAACGGTGTC
ACCGTTGTTGCCTCCTTCACTGGTACCAAGACTGGTATCGGTGGCTACG
TCTCCACCGACAACACCAACAAGGAGATCGTCCTTTCTTTCCGTGGCAG
CACCAACATCCGCAACTGGCTGACCAACCTGGACTTCGGCCAGGATGAC
TGCTCTCTGACCTCCGGCTGCGGTGTCCACTCCGGTTTCCAGCGTGCCT
GGGAGGAGATTGCCGACAACCTGACCGCTGCTGTTGCCAAGGCCAAGAC
TGCCAACCCCGACTACAAGGTTGTTGCCACTGGCCACTCCCTGGGTGGT
GCTGTTGCCACCCTGGCTGGTGCCAACCTCCGTGCTGCTGGTACCCCCC
TCGACATCTACACCTACGGCTCTCCCCGTGTCGGCAACGCCGAGCTTGC
TGAGTTCATCTCCAACCAGACTGGTGGTGAGTTCCGTGTCACCCACGGT
GATGACCCCGTCCCCCGTCTTCCTCCTCTGATCTTCGGCTACCGCCACA
CCTCCCCCGAGTACTGGCTCGATGGCAGCGGTGGTGACAAGATCAACTA
CACCATCAACGACATCAAGGTCTGCGAGGGTGCTGCCAACCTGCAGTGC
AACGGTGGTACCCTGGGCCTCGACATTGCTGCTCACCTGCACTACTTCC
AGGCCACTGGTGCCTGCAACGCCGGTGGTTTCAGCTGGCGCCGCTACCG
CTCTGCTGAGAGCGTTGACAAGCGTGCCACCATGACTGATGCTGAGCTC
GAGAAGAAGCTCAACAGCTACGTGCAGATGGACAAGGAGTACGTCAAGA
ACAACCAGGCTCGCTCCTAAACGAGACC

In addition, polypeptide variants named M15_002, M15_004, M15_006, M15_008, M15_055, M15_056, M15_057 and M15_066 were designed having modifications in the loop (linker) of the KEX2 protease cleavage site and adjacent C-terminal pro-peptide, amino acids 305 to 320 of SEQ ID NO: 1. The amino acids RRYRSAESVDKRATMT were replaced by the sequences listed in Table 1, resulting in polypeptides with amino acid sequences as set forward in SEQ ID NO: 3-10.

TABLE 1

| | Sequence of loop | | | | |
|---|---|---|---|---|---|
| | (linker) replacing | | | | SEQ ID NO: of |
| | amino acids | SEQ ID NO: | SEQ ID NO: | | polynucleotide |
| Variant | 305-320 of M15 | of the | of loop | | encoding the |
| name | (SEQ ID NO: 1) | polypeptide | (linker) | Vector | polypeptide |
| M15 | RRYRSAESVDKRA | 1 | 19 | PGBTOP- | 2 |
| | TMT | | | M15 | |
| M15_002 | QRYRSAESVDKRA | 3 | 20 | PGBTOP- | 11 |
| | TMT | | | M15_02 | |
| M15_004 | ARYRSAESVDKRAT | 4 | 21 | PGBTOP- | 12 |
| | MT | | | M15_04 | |
| M15_006 | RQYRSAESVDKRA | 5 | 22 | pGBTOP- | 13 |
| | TMT | | | M15_06 | |
| M15_008 | RAYRSAESVDKRAT | 6 | 23 | PGBTOP- | 14 |
| | MT | | | M15_08 | |
| M15_055 | KEREEDIS | 7 | 24 | PGBTOP- | 15 |
| | | | | M15_55 | |
| M15_056 | RQREEEIS | 8 | 25 | PGBTOP- | 16 |
| | | | | M15_56 | |
| M15_057 | KPHMS | 9 | 26 | PGBTOP- | 17 |
| | | | | M15_57 | |
| M15_066 | RSRQEDIS | 10 | 27 | PGBTOP- | 18 |
| | | | | M15_66 | |

The codon optimized DNA sequences for expression of the genes encoding the polypeptides polypeptide variants named M15_002, M15_004, M15_006, M15_008, M15_055, M15_056, M15_057 and M15_066 in A. niger were decorated with Bsal type II restriction enzyme sites to enable subcloning in pGBTOP-18 (FIG. 1).

The DNA fragments SEQ ID NO: 2 and SEQ ID NO: 11-18 were cloned into pGBTOP-18 thru repetitive steps of Bsal digestion and ligation (GoldenGate cloning method (New England Biolabs), according to standard procedure. The resulting vectors containing the lipolytic enzyme expression cassettes under control of the glucoamylase promoter resulted in vectors pGBTOP-M15 and pGBTOP-M15_02 through pGBTOPM15_66, respectively.

Subsequently, A. niger GBA 306 was transformed with pGBTOP-M15, pGBTOP-M15_02 through pGBTOP-M15_66 in a co-transformation protocol with pGBAAS-4, with strain and methods as described in WO2011/009700 and references therein and selected on acetamide containing media and colony purified according to standard procedures. Transformation and selection were performed as described in WO98/46772 and WO99/32617. Eight transformants expressing the same gene variant were selected as representative transformants, and further replica-plated to obtain single strain inoculum spore suspensions.

### Example 2: Fermentation of Lipase Expressing A. niger Strains

Fresh A. niger spores from the parents strain GBA306 and the M15 and M15_02 through M15_66 expressing strains GBA306-M15 and GBA306-M15_02 through GBA306-M15_66 were prepared and used to generate sample material by cultivation of the strains in 24 deep well-plates containing 3 ml fermentation medium (15% w/v maltose, 6% w/v bacto-soytone, 1.5% w/v (NH₄)₂SO₄, 0.1% w/v NaH₂PO₄.H₂O, 0.1% w/v MgSO₄.7H₂O, 0.1% w/v L-arginine, 8‰ w/v Tween-80, 2‰ w/v Basildon, 2% w/v MES, pH 5.1). After 6 days of cultivation at 34° C., 550 rpm and 80% humidity in a Microton incubator shaker (Infors AG, Bottmingen, Switzerland) 1.5 mL samples were taken, the mycelium was separated from the supernatant by centrifugation for 30 min at 4000 g and the supernatants were stored at −20° C. until further analyses. The enzyme expression levels in the supernatants were compared by loading equal volumes of the same dilution of the stored variant and control supernatant samples on the same Coomassie-stained SDS PAGE gel, and then comparing the intensity of bands between the 28 to 49 kDa ladder bands to the bands of the control sequence SEQ ID 1(Table 2). The SeeBlue® Plus2 Pre-Stained Standard of Thermo Fischer was used as a pre-stained ladder in this experiment.

As can clearly be observed, the absence of the parent KEX2 protease cleavage site in the lipase polypeptide produced surprisingly resulted in all cases in a significant increase in yield of the polypeptide produced when compared to the reference (M15) polypeptide with KEX2 protease cleavage site present.

TABLE 2

| | | Enzyme expression |
|---|---|---|
| Strain | Enzyme | on SDS-PAGE |
| GBA306 | − | − |
| GBA306-M15 | SEQ ID NO: 1 | + |
| GBA306-M15_002 | SEQ ID NO: 3 | ++ |
| GBA306-M15_004 | SEQ ID NO: 4 | ++ |
| GBA306-M15_006 | SEQ ID NO: 5 | ++ |
| GBA306-M15_008 | SEQ ID NO: 6 | ++ |
| GBA306-M15_055 | SEQ ID NO: 7 | ++++ |
| GBA306-M15_056 | SEQ ID NO: 8 | ++++ |
| GBA306-M15_057 | SEQ ID NO: 9 | ++++ |
| GBA306-M15_066 | SEQ ID NO: 10 | ++++ |

### Example 3: Cloning and Expression Second Enzyme LPV06 and Lipolytic Enzyme Variants

A second parent polypeptide (also referred to as LPV06) SEQ ID NO: 28 comprises a signal peptide (amino acids 1-15), an N-terminal pro-peptide (amino acids 16-30), a mature protein (amino acids 31-304) having lipolytic activity and a Kex2 protease cleavage site and C-terminal pro-peptide (amino acids 305-346). The mature proteins of M15 and LPV06 are 77% identical.

Polypeptide variants of LPV06 named R3_072 and R3_073 were designed having modifications in the loop (linker) of the KEX2 protease cleavage site and adjacent C-terminal pro-peptide amino acids 305 to 320 of SEQ ID NO: 28. The amino acids RRYRSAESVDKRATMT (SEQ ID NO: 19) were replaced by sequences listed in Table 3, resulting in polypeptides with amino acid sequences as set forward in SEQ ID NO: 29-30.

The designed polypeptides were back-translated into codon optimized DNA resulting in nucleotide sequences SEQ ID NO: 31, 32 and 33. The constructs were cloned into pGBTOP-18 and transformed to Aspergillus niger as described in examples 1 and 2 herein.

TABLE 3

| | Sequence of loop | | | | |
|---|---|---|---|---|---|
| | (linker) replacing | | SEQ ID | | SEQ ID NO: of |
| | amino acids | SEQ ID NO: | NO: | | polynucleotide |
| Variant | 305-320 of LPV06 | of the | of loop | | encoding the |
| name | (SEQ ID NO: 28) | polypeptide | (linker) | Vector | polypeptide |
| LPV06 | RRYRSAESVDKRATMT | 28 | 19 | PGBTOP- | 31 |
| | | | | R3_79 | |
| R3_072 | KEREEDIS | 29 | 24 | PGBTOP- | 32 |
| | | | | R3_72 | |
| R3_073 | KPHMS | 30 | 26 | pGBTOP- | 33 |
| | | | | R3_73 | |

The enzyme-expressing strains were cultivated in 24 deep-well plates containing 3 ml fermentation medium as described in example 2. After 6 days of cultivation the enzyme expression levels in the supernatants were compared on SDS-PAGE as described above in Example 2, where the control sequence is now SEQ ID 28 (Table 4).

TABLE 4

| | | Enzyme expression |
|---|---|---|
| Strain | Enzyme | on SDS-PAGE |
| GBA306-LPV06 | SEQ ID NO: 28 | + |
| GBA306-R3_72 | SEQ ID NO: 29 | +++ |
| GBA306-R3_73 | SEQ ID NO: 30 | +++ |

As can clearly be observed, the absence of the parent KEX2 protease cleavage site in the lipase polypeptide produced (R3_72 & R3_73) surprisingly resulted in both cases in a significant increase in yield of the polypeptide produced when compared to the reference (LPV06) polypeptide with KEX2 protease cleavage site present.

### Example 4: Phospholipase Enzyme (PLE) Activities of Lipase Expressing A. niger Strains as a Result of pH and Diacetate Treatment

The phospholipase enzyme (PLE) activity levels and amount of protein in selected broth samples as well as supernatant samples as described in Example 2 and Example 3 were determined for variant and control supernatant samples after growth and fermentation at shake flask scale as described above. Additionally, a pH treatment and diacetate treatment were applied on selected lipase enzyme variants in broth samples as well as supernatant samples, after which the same protein and phospholipase (PLE) activity assays were performed.

As can clearly be observed from Table 2 and 4 (Example 2 and 3), the absence of the parent KEX2 protease cleavage site resulted in a significant increase in productivity of the variant lipolytic polypeptide resulting from SDS-PAGE analysis. The increased protein levels for lipolytic enzyme variants with absence of the KEX2 protease cleavage site were paralleled by an increased lipase activity level for those variants in both broth and supernatant when compared to the M15 reference (data not shown). To be able to compare activity and protein levels in each sample throughout time, each “End of Fermentation” sample for each lipolytic enzyme variant was set at 100% for both PLE activity as well as for Bradford protein concentration. Surprisingly, the absence of the parent KEX2 protease cleavage site in the lipolytic enzyme variants resulted in increased PLE lipase activity levels after incubation/applying a diacetate treatment (Table 5) at pH 5.0. The activity levels cannot be explained by the different protein content in the different samples. Where the reference M15 lost 60%-70% of PLE activity in broth and supernatant upon diacetate treatment, all variants with absence of the parent KEX2 protease cleavage site lost less PLE activity (M15_002, M15_057) or even gained PLE activity level (M15_055, M15_066). The same holds for LPV06 lipase reference, where variant enzyme LPV06-R3_72 surprisingly shows a gained PLE activity level upon diacetate treatment (Table 5) where the reference lost PLE activity upon diacetate treatment.

In a next experiment, the M15 reference enzyme with a KEX2 cleavage site was compared with one lipolytic enzyme variant with absent KEX2 protease cleavage site (M15_055) in broth background under various treatments at the end of fermentation. As can be learned from Table 6, the absence of the KEX2 protease cleavage site results in increased PLE lipase activity levels upon various treatments after fermentation, such as incubation at pH 5.0, and diacetate treatment conditions (time variations) as described in Table 6.

The pH treatment and diacetate treatment as performed are examples of typical conditions and methods that can be applied for production and purification of enzymes at large scale using broth and/or cell-free supernatant in the downstream process and/or in killing-off the production strain. Therefore, the absence of the KEX2 protease cleavage site in a lipolytic enzyme is a means to efficiently produce more lipase polypeptide and/or produce more active lipase enzyme as measured by an activity assay.

TABLE 5
Relative (%) phospholipase (PLE) enzymatic activities and relative (%) protein
concentrations in various broth and supernatant samples after shake flask fermentation
and diacetate treatment (Diacetate treatment for the different strains/samples was performed
with 15 g/kg sodium diacetate at pH 5.0 and 30 C. for 6 hours).

| | | Broth | | | | Supernatant | | | |
|---|---|---|---|---|---|---|---|---|---|
| | | Phospholipase | | Protein (% | | Phospholipase | | Protein (% | |
| | Enzyme | activity (% PLE) | | Bradford) | | activity (% PLE) | | Bradford) | |
| Strain/enzyme | SEQ ID | End of | Diacetate | End of | Diacetate | End of | Diacetate | End of | Diacetate |
| sample | NO: | Fermentation | treatment | Fermentation | treatment | Fermentation | treatment | Fermentation | treatment |
| GBA306 | — | — | — | 6 | 2 | — | — | 3 | 3 |
| GBA306-M15 | 1 | 100 * | 42 | 100 * | 79 | 96 | 31 | 63 | 36 |
| GBA306- | 3 | 100 * | 71 | 100 * | 73 | 114 | 65 | 55 | 41 |
| M15_002 | | | | | | | | | |
| GBA306- | 7 | 100 * | 255 | 100 * | 86 | 65 | 254 | 66 | 65 |
| M15_055 | | | | | | | | | |
| 100 *GBA306- | 9 | 100 * | 95 | 100 * | 81 | 68 | 34 | 81 | 65 |
| M15_057 | | | | | | | | | |
| GBA306- | 10 | 100 * | 110 | 100 * | 107 | 40 | 29 | 17 | 14 |
| M15_066 | | | | | | | | | |
| GBA306- | 28 | 100 * | 82 | 100 * | 74 | 82 | 71 | 71 | 56 |
| LPV06 | | | | | | | | | |
| GBA306- | 29 | 100 * | 185 | 100 * | 82 | 71 | 182 | 73 | 71 |
| R3_72 | | | | | | | | | |

* indicates the reference value set at 100% for that strain/enzyme sample

TABLE 6
Relative (%) phospholipase (PLE) enzymatic activities of broth
samples as indicated (corrected for dilution of treatment) after shake
flask fermentation and end-of-fermentation pH adjustment (pH = 5)
and diacetate treament with pH adjustment (Diacetate treatment for
the different strains was tested with 15 g/kg sodium diacetate at pH
5.0 and 30° C. for various hours, as indicated).

| | pH adjustment | | | Diacetate treatment | | |
|---|---|---|---|---|---|---|
| | Phospholipase activity (% PLE) | | | Phospholipase activity (% PLE) | | |
| Strain/enzyme | 0 g/kg diacetate | | | 15 g/kg diacetate | | |
| sample | 0 hrs | 8 hrs | 24 hrs | 0 hrs | 8 hrs | 24 hrs |
| GBA306-M15 broth | 100 * | 63 | 34 | 100 * | 41 | 22 |
| GBA306-M15_055 broth | 100 * | 116 | 144 | 100 * | 275 | 297 |

* indicates the reference value set at 100% for that strain/enzyme sample

1. A variant of a parent polypeptide, wherein a difference between the variant polypeptide and the parent polypeptide is that a KEX2 protease cleavage site which is present in the parent polypeptide, is absent in the variant polypeptide, wherein optionally the KEX2 protease cleavage site comprises and/or consists of the amino acids KK, RR, RK or KR,
wherein the parent polypeptide has at least 80% sequence identity with the amino acid sequence as set forward in SEQ ID NO: 34 or
wherein the parent polypeptide has at least 80% sequence identity with the amino acid sequence as set forward in SEQ ID NO: 34 and wherein the variant polypeptide further comprises at least one substitution of an amino acid residue at a position corresponding to any of positions 113, 121, 138, 141, 179, 282, 284, 286, 295 of SEQ ID NO: 34.

2. A variant polypeptide according to claim 1, wherein the KEX2 protease cleavage site is present immediately before a pro-peptide within the parent polypeptide and wherein, optionally, the pro-peptide having the KEX2 protease cleavage site immediately before said pro-peptide, is located at the C-terminus of the parent polypeptide.

3. A variant polypeptide according to claim 1, wherein, when produced under the same circumstances as the parent polypeptide, the variant polypeptide has a higher yield compared to the parent polypeptide, and/or wherein the variant polypeptide and parent polypeptide have lipolytic activity and are optionally EC 3.1.1.3 triacylglycerol lipases.

4. A variant polypeptide according to claim 1, wherein the KEX2 protease cleavage site is removed by:
(a) deletion or substitution of at least one amino acid residue of the KEX2 protease cleavage site, or
(b) replacement of the KEX2 protease cleavage site and optionally one or more contiguous amino acids adjacent to the KEX2 protease cleavage site by an amino acid motif not containing a KEX2 protease cleavage site.

5. A variant polypeptide according to claim 4, wherein the linker motif adjacent to the C-terminal pro-peptide is replaced by an amino acid motif selected from the group consisting of SEQ ID NO: 20, 21, 22, 23, 24, 25, 26 and 27 and/or wherein the linker motif adjacent to the C-terminal pro-peptide in the parent polypeptide has the amino acid sequence as said forward in SEQ ID NO: 19.

6. A variant polypeptide according to claim 1, wherein the variant polypeptide has an amino acid sequence selected from the group consisting of SEQ ID NO: 3, 4, 5, 6, 7, 8, 9 and 10 and optionally comprises at least one substitution of an amino acid residue at a position corresponding to any of the positions 113, 121, 138, 141, 179, 282, 284, 286, 295.

7. A polynucleotide encoding the variant polypeptide according to claim 1, wherein the polynucleotide optionally is codon optimized.

8. A recombinant host cell comprising the polynucleotide according to claim 7.

9. A process for producing a variant polypeptide according to claim 1 comprising cultivating the host cell under conditions conducive to production of the variant polypeptide and recovering the variant polypeptide.

10. A process for producing a mature polypeptide having lipolytic activity comprising cultivating the host cell of claim 9 under conditions conducive to production of a variant polypeptide, recovering the variant polypeptide and activating the variant polypeptide to obtain said mature polypeptide having lipolytic activity.

11. A dough comprising the variant polypeptide according to claim 1.

12. A process for production of a baked product, which process comprises baking the dough according to claim 11.

13. A baked product obtainable by the process according to claim 12.

14. A baked product according to claim 13, wherein the product is a bread, a cake or baked product prepared from a laminated dough.

15. A dough according to claim 11, wherein the dough comprises a wholemeal flour and/or a flour comprising a free fatty acid content of between 0.01 to 0.8 w/w %.
